(19) United States
(12) Patent Application Publication
(10) Pub. No.: US 2023/0417767 A1
(43) Pub. Date: Dec. 28, 2023

(54) COMPOSITIONS AND METHODS FOR THE DIAGNOSIS AND TREATMENT OF AGE-RELATED MACULAR DEGENERATION

(71) Applicant: Georgetown University, Washington, DC (US)

(72) Inventor: Nady Golestaneh, Bethesda, MD (US)

(21) Appl. No.: 18/138,058

(22) Filed: Apr. 22, 2023

Related U.S. Application Data
(63) Continuation of application No. 16/075,932, filed on Aug. 6, 2018, now abandoned.
(60) Provisional application No. 62/292,267, filed on Feb. 6, 2016.

(51) Int. Cl. G01N 33/68; A61K 31/216; A61K 31/4184; A61K 31/4245; A61K 31/192; A61K 31/41; A61K 31/55; A61K 31/194; A61K 31/195; A61K 31/4178; C12Q 1/6883; G01N 33/50

(52) U.S. Cl. CPC G01N 33/6893; A61K 31/216; A61K 31/4184; A61K 31/4245; A61K 31/192; A61K 31/41; A61K 31/55; A61K 31/194; A61K 31/195; A61K 31/4178; C12Q 1/6883; G01N 33/5023; G01N 2800/164; C12Q 2600/158; G01N 2333/705; G01N 2333/91142; G01N 2333/978

(57) ABSTRACT

The present invention is related to diagnostic, treatment and compound screening methods related to dry age-related macular degeneration (dry AM D). In select embodiments, the methods comprise determining expression or activity levels of NAD-dependent deacetylase sirtuin-1 (SI RT-1), AM P-activated protein kinase (AM PK), poly(adenosine diphosphate ribose) polymerase-2 (PARP2), peroxisome proliferator-activated receptor-gamma coactivator 1-alpha (PGC-Iα) and/or mRNA levels of RAC-gamma serine/threonine-protein kinase (AKT3). In general, higher levels of PARP2, lower levels of PGC-Iα or AKT3 and/or higher acetylation levels of PGC-Iα in the samples are indicative that the subject or cells from which the samples are obtained are susceptible or are suffering from dry AMD.

### CROSS-REFERENCE TO RELATED APPLICATIONS

The present application is a continuation of U.S. application Ser. No. 16/075,932, filed on Aug. 6, 2018, which is a national stage entry under 35 U.S.C. 371 of International Application No. PCT/US2017/016655, filed on Feb. 6, 2017, which claims priority to U.S. Provisional Application Ser. No. 62/292,267, filed on Feb. 6, 2016, the entire contents of each application of which are hereby incorporated by reference herein.

### BACKGROUND OF THE INVENTION

### Field of the Invention

The present invention is related to diagnostic, treatment and compound screening methods related to dry age-related macular degeneration (dry AMD). In select embodiments, the methods comprise determining expression or activity levels of NAD-dependent deacetylase sirtuin-1 (SIRT-1), AMP-activated protein kinase (AMPK), poly(adenosine diphosphate ribose) polymerase-2 (PARP2), peroxisome proliferator-activated receptor-gamma coactivator 1-alpha (PGC-1α) and/or mRNA levels of RAC-gamma serine/threonine-protein kinase (AKT3). In general, higher levels of PARP2, lower levels of SIRT-1, AMPK, PGC-1α or AKT3 and/or higher acetylation levels of PGC-1α in the samples are indicative that the subject or cells from which the samples are obtained are susceptible or are suffering from dry AMD.

### Background of the Invention

Age-related macular degeneration (AMD) is the leading cause of vision loss among people over the age of 50 in developed countries worldwide. AMD affects approximately 30-50 million people, (Gehrs et al., 2006; Klein et al., 2006; Klein et al., 2011) and its prevalence is expected to double by 2050 (Rein et al., 2009).

AMD initially affects the retinal pigment epithelium (RPE), a monolayer of pigmented and polarized central nervous system (CNS) tissue, and over time, leads to secondary loss of photoreceptor cells (Bok, 1993; Boulton and Dayhaw-Barker, 2001; Gehrs et al., 2006). AMD is a progressive, multifactorial disease (Rein et al., 2009) and its pathogenesis remains largely elusive. Mounting evidence suggests a complex interaction of genetic, environmental, and metabolic factors contributing to the pathology of AMD (Nowak, 2006). Impaired RPE function in “dry AMD” causes the formation of abnormal extracellular deposits called drusen (Abdelsalam et al., 1999) that accumulate between the RPE and Bruch's membrane (BM). The wet form of AMD involves choroidal neovascularization followed by formation of a disciform scar (Ferris et al., 1984).

The RPE plays important roles in retinal homeostasis. It functions as a retinal blood barrier, a source of polarized growth factor release and transporter of ions, water, and metabolic products from the subretinal space to the blood (Dornonville de la Cour, 1993; Hamann, 2002). The RPE delivers blood-derived nutrients to photoreceptors, absorbs light, and performs phagocytosis of the outer segments of photoreceptors (Strauss, 2005). Numerous studies have reported age-related physiological changes in RPE (Kozlowski, 2012), including mitochondrial DNA damage (Lin et al., 2011), accumulation of lipofuscin (Schmitz-Valckenberg et al., 2009), elevated amyloid β production (Wang et al., 2012), enhanced tissue factor (TF) expression (Cho et al., 2011), increased acidic beta-galactosidase activity suggestive of lysosomal dysfunction (Matsunaga et al., 1999; Kurz et al., 2000), and altered expression of RPE structural proteins (Shelton et al., 1999; Gu et al., 2012). Despite recent progress, the mechanisms impaired in human RPE that contribute to AMD have not been elucidated.

Autophagy is a lysosome-mediated degradation process for non-essential or damaged cellular constituents to supply the cell with energy and to maintain homeostasis (De Duve, 1963; De Duve and Wattiaux, 1966; Finn and Dice, 2006). Recently, much interest has focused on the role of autophagy in health and disease (Czaja, 2010; Wang et al., 2010; Rubinsztein et al., 2011; Orenstein et al., 2013). Moreover, lipid droplets have been identified as an autophagy substrate (Singh et al., 2009) and the impact of autophagy on mitochondrial function has been discussed (Zhang et al., 2007; Lee et al., 2012). The latter is particularly relevant to degenerative diseases, such as AMD, in which oxidative stress occurs over time. Despite multiple reviews proposing a role for autophagy in AMD (Ambati and Fowler, 2012; Kinnunen et al., 2012; Mitter et al., 2012), direct evidence for altered autophagy in the pathophysiology of AMD has not yet been provided. In addition, dysfuncational autophagy has recently been reported in RPE from AMD donor eyes, as well as decreased PGC-1α expression and SIRT1 protein levels in iPSC-derived RPE.

### SUMMARY OF THE INVENTION

The present invention provides for methods of determining if a subject is at risk of developing age-related macular degeneration (AMD) of the “dry type,” with the methods comprising determining expression or activity levels of peroxisome proliferator-activated receptor-gamma coactivator 1-alpha (PGC-1α) in a sample obtained from the subject, and comparing the determined expression or activity levels of PGC-1α in the subject with the expression or activity levels obtained from subjects determined to define normal expression or activity levels of PGC-1α to determine if the subject's expression or activity levels of PGC-1α are altered compared to normal expression or activity levels of PGC-1α. A reduction in the expression or activity levels of PGC-1α in the sample compared to normal levels is indicative that the subject is at risk of developing dry AMD.

The present invention also provides for methods of determining if a subject is at risk of developing dry type AMD with the methods comprising determining the levels of acetylated PGC-1α in a sample obtained from the subject, and comparing the determined levels of acetylated PGC-1α in the subject with the levels of acetylated PGC-1α obtained from subjects determined to define normal levels of acetylated PGC-1α to determine if the subject's levels of acetylated PGC-1α are altered compared to normal levels of acetylated PGC-1α. An increase in the levels of acetylted PGC-1α in the sample compared to normal levels is indicative that the subject is at risk of developing dry AMD.

The present invention also provides methods of determining if a subject is at risk of developing dry AMD, with the methods comprising determining mRNA levels of RAC-gamma serine/threonine-protein kinase (AKT3) in a sample obtained from the subject, and comparing the determined mRNA levels of AKT3 in the subject with the mRNA levels obtained from subjects determined to define normal mRNA levels of AKT3 to determine if the subject's mRNA levels of AKT3 are altered compared to normal mRNA levels of AKT3. A reduction in the mRNA levels of AKT3 in the sample compared to normal mRNA levels is indicative that the subject is at risk of developing dry AMD.

The present invention also provides methods of determining if a subject is at risk of developing dry AMD, with the methods comprising determining expression or activity levels of poly(adenosine diphosphate ribose) polymerase 2 (PARP2) in a sample obtained from the subject, and comparing the determined expression or activity levels of PARP2 in the subject with the expression or activity levels obtained from subjects determined to define normal expression or activity levels of PARP2 to determine if the subject's expression or activity levels of PARP2 are altered compared to normal expression or activity levels of PARP2. An increase in the expression or activity levels of PARP2 in the sample compared to normal levels is indicative that the subject is at risk of developing dry AMD.

The present invention also provides methods of determining if a subject is at risk of developing dry AMD, with the methods comprising determining expression or activity levels of NAD-dependent deacetylase sirtuin-1 (SIRT-1) in a sample obtained from the subject, and comparing the determined expression or activity levels of SIRT-1 in the subject with the expression or activity levels obtained from subjects determined to define normal expression or activity levels of SIRT-1 to determine if the subject's expression or activity levels of SIRT-1 are altered compared to normal expression or activity levels of SIRT-1. A reduction in the expression or activity levels of SIRT-1 in the sample compared to normal levels is indicative that the subject is at risk of developing dry AMD.

The present invention also provides methods of monitoring the progression of dry AMD in a subject, with the methods comprising analyzing at least two samples from the subject with each sample taken at different time points to determine expression or activity levels of PGC-1α, and comparing the expression or activity levels of the subject's PGC-1α over time to determine if expression or activity levels of PGC-1α are changing over time. A decrease in the subject's expression or activity levels of PGC-1α over time is indicative that the subject's risk of suffering from dry AMD is increasing over time.

The present invention also provides methods of determining if a subject is at risk of developing dry AMD, with the methods comprising determining expression or activity levels of AMP-activated protein kinase (AMPK) in a sample obtained from the subject, and comparing the determined expression or activity levels of AMPK in the subject with the expression or activity levels obtained from subjects determined to define normal expression or activity levels of AMPK to determine if the subject's expression or activity levels of AMPK are altered compared to normal expression or activity levels of AMPK. A reduction in the expression or activity levels of AMPK in the sample compared to normal levels is indicative that the subject is at risk of developing dry AMD.

The present invention also provides methods of monitoring the progression of dry AMD in a subject, with the methods comprising analyzing at least two samples from the subject with each sample taken at different time points to determine levels of acetylated PGC-1α, and comparing the levels of acetylated PGC-1α over time to determine if levels of acetylated PGC-1α are changing over time. An increase in the subject's levels of acetylated PGC-1α over time is indicative that the subject's risk of suffering from dry AMD is increasing over time.

The present invention also provides methods of monitoring the progression of dry AMD in a subject, with the methods comprising analyzing at least two samples from the subject with each sample taken at different time points to determine mRNA levels of AKT3, and comparing the mRNA levels of the subject's AKT3 over time to determine if mRNA levels of AKT3 are changing over time. A decrease in the subject's mRNA levels of AKT3 over time is indicative that the subject's risk of suffering from dry AMD is increasing over time.

The present invention also provides methods of monitoring the progression of dry AMD in a subject, with the methods comprising analyzing at least two samples from the subject with each sample taken at different time points to determine expression or activity levels of PARP2, and comparing the expression or activity levels of the subject's PARP2 over time to determine if expression or activity levels of PARP2 are changing over time. An increase in the subject's expression or activity levels of PARP2 over time is indicative that the subject's risk of suffering from dry AMD is increasing over time.

The present invention also provides methods of monitoring the progression of dry AMD in a subject, with the methods comprising analyzing at least two samples from the subject with each sample taken at different time points to determine expression or activity levels of SIRT-1, and comparing the expression or activity levels of the subject's SIRT-1 over time to determine if expression or activity levels of SIRT-1 are changing over time. A decrease in the subject's expression or activity levels of SIRT-1 over time is indicative that the subject's risk of suffering from dry AMD is increasing over time.

The present invention also provides methods of monitoring the progression of a treatment of dry AMD in a subject, with the methods comprising analyzing at least two samples from a subject undergoing treatment for dry AMD with each sample taken at different time points to determine expression or activity levels of PGC-1α at the different time points, and comparing the values of the subject's expression or activity levels of PGC-1α over time to determine if the subject's expression or activity levels of PGC-1α are changing over time in response to the treatment. A lack of change or a further deviation from normal expression or activity levels of PGC-1α in the subject's expression or activity levels of PGC-1α is indicative that the treatment for dry AMD is not effective, and an approximation of the subject's expression or activity levels of PGC-1α over time towards normal expression or activity levels of PGC-1α is indicative that the treatment for dry AMD is effective in treating dry AMD in the subject.

The present invention also provides methods of monitoring the progression of a treatment of dry AMD in a subject, with the methods comprising analyzing at least two samples from a subject undergoing treatment for dry AMD with each sample taken at different time points to determine levels of acetylated PGC-1α at the different time points, and comparing the values of the subject's levels of acetylated PGC-1α over time to determine if the subject's levels of acetylated PGC-1α are changing over time in response to the treatment. A lack of change or a further deviation from normal levels of acetylated PGC-1α in the subject's levels of acetylated PGC-1α is indicative that the treatment is not effective in treating dry AMD in the subject, and an approximation of the subject's levels of acetylated PGC-1α over time towards normal levels of acetylated PGC-1α is indicative that the treatment is effective in treating dry AMD in the subject.

The present invention also provides methods of monitoring the progression of a treatment of dry AMD in a subject, with the methods comprising analyzing at least two samples from a subject undergoing treatment for dry AMD with each sample taken at different time points to determine mRNA levels of AKT3 at the different time points, and comparing the values of the subject's mRNA levels of AKT3 over time to determine if the subject's mRNA levels of AKT3 are changing over time in response to the treatment. A lack of change or a further deviation from normal mRNA levels of AKT3 in the subject's mRNA levels of AKT3 is indicative that the treatment is not effective in treating dry AMD in the subject, and an approximation of the subject's mRNA levels of AKT3 over time towards normal mRNA levels of AKT3 is indicative that the treatment is effective in treating dry AMD in the subject.

The present invention also provides methods of monitoring the progression of a treatment of dry AMD in a subject, with the methods comprising analyzing at least two samples from a subject undergoing treatment for dry AMD with each sample taken at different time points to determine expression or activity levels of PARP2 at the different time points, and comparing the values of the subject's expression or activity levels of PARP2 over time to determine if the subject's expression or activity levels of PARP2 are changing over time in response to the treatment. A lack of change or a further deviation from normal expression or activity levels of PARP2 in the subject's expression or activity levels of PARP2 is indicative that the treatment for dry AMD is not effective, and an approximation of the subject's expression or activity levels of PARP2 over time towards normal expression or activity levels of PARP2 is indicative that the treatment for dry AMD is effective in treating dry AMD in the subject.

The present invention also provides methods of monitoring the progression of a treatment of dry AMD in a subject, with the methods comprising analyzing at least two samples from a subject undergoing treatment for dry AMD with each sample taken at different time points to determine expression or activity levels of SIRT-1 at the different time points, and comparing the values of the subject's expression or activity levels of SIRT-1 over time to determine if the subject's expression or activity levels of SIRT-1 are changing over time in response to the treatment. A lack of change or a further deviation from normal expression or activity levels of SIRT-1 in the subject's expression or activity levels of SIRT-1 is indicative that the treatment for dry AMD is not effective, and an approximation of the subject's expression or activity levels of SIRT-1 over time towards normal expression or activity levels of SIRT-1 is indicative that the treatment for dry AMD is effective in treating dry AMD in the subject.

The present invention also provides methods of screening a test compound for its effectiveness in treating dry AMD, with the methods comprising administering the test compound to abnormal RPE cells and determining expression or activity levels of PGC-1α in the abnormal RPE cells in response to the administration of the target compound. An increase in expression or activity levels of PGC-1α indicates that the target compound may be effective in treating dry AMD.

The present invention also provides methods of screening a test compound for its effectiveness in treating dry AMD, with the methods comprising administering the test compound to abnormal RPE cells and determining levels of acetylated PGC-1α in the abnormal RPE cells in response to the administration of the target compound. A decrease in levels of acetylated PGC-1α indicates that the target compound may be effective in treating dry AMD.

The present invention also provides methods of screening a test compound for its effectiveness in treating dry AMD, the method comprising administering the test compound to abnormal RPE cells and determining mRNA levels of AKT3 in the abnormal RPE cells in response to the administration of the target compound. An increase in mRNA levels of AKT3 indicates that the target compound may be effective in treating dry AMD.

The present invention also provides methods of screening a test compound for its effectiveness in treating dry AMD, with the methods comprising administering the test compound to abnormal RPE cells and determining expression or activity levels of PARP2 in the abnormal RPE cells in response to the administration of the target compound. A decrease in expression or activity levels of PARP2 indicates that the target compound may be effective in treating dry AMD.

The present invention also provides methods of screening a test compound for its effectiveness in treating dry AMD, with the methods comprising administering the test compound to abnormal RPE cells and determining expression or activity levels of SIRT-1 in the abnormal RPE cells in response to the administration of the target compound. An increase in expression or activity levels of SIRT-1 indicates that the target compound may be effective in treating dry AMD.

The present invention also relates to methods of treating dry AMD in a subject in need of treatment thereof, with the method comprising administering a compound that induces expression or activity of PGC-1α in retinal pigment epithelial cells.

The present invention also relates to methods of treating dry AMD in a subject in need of treatment thereof, with the method comprising administering a compound that reduces levels of acetylated PGC-1α in retinal pigment epithelial cells.

The present invention also relates to methods of treating dry AMD in a subject in need of treatment thereof, with the method comprising administering a compound that induces expression of or activity AKT3 in retinal pigment epithelial cells.

The present invention also relates to methods of treating dry AMD in a subject in need thereof, with the methods comprising inhibiting the expression or activity of PARP2 in retinal pigment epithelial cells.

The present invention also relates to methods of treating dry AMD in a subject in need thereof, with the methods comprising increasing the expression or activity of SIRT-1 in retinal pigment epithelial cells.

### BRIEF DESCRIPTION OF THE DRAWINGS

FIGS. 1A-1H depict isolation and characterization of RPE from donors. FIGS. 1A-1F show isolated RPE from AMD and control donors expressing the epithelial markers ZO-1, Bestrophin and CRALBP. A representative image of immunostaining is shown for each group. Bar represents 100 μm. FIG. 1G shows gene expression analysis by qRT-PCR confirming the expression of RPE marker genes in the RPE isolated from donors (controls 6, 10, 23 and 25; AMD 9, 17, 19 and 32). FIG. 1H shows gene expression analysis of AMD associated genes in AMD and normal RPE. * p<0.05

FIGS. 2A-2C depict the differentiation and characterization of generated iPSC-RPE. FIG. 2A shows immunostaining of the iPSC-RPE cultured on transwells for 4 weeks with ZO-1 antibody, RPE65, Occludin, and Bestrophin. Scale bar represent 100 μm. FIG. 2B shows a graph illustrating RPE specific gene expressions in differentiated iPSC-RPE cell lines. FIG. 2C shows RPE specific gene expression in native RPE from which the iPSC-RPE are generated. Relative expression of each gene to GAPDH is compared to its relative expression level in control iPSC-RPE.

FIG. 3 depicts the genotyping of AMD and control RPE. The 5 clinically diagnosed AMD donors and 5 clinically normal donors (control) from which primary RPE cultures were established, and iPSC-RPE were generated. Genotyping of a dry AMD patient skin fibroblasts from which iPSC-RPE were generated (005BF). Genotyping data for known AMD-associated Single Nuclear Polymorphisms showing the haplotypes of each donor, carrying risk or protective alleles.

FIGS. 4A-4F depict electron microscopy images of normal (FIGS. 4A, 4C, and 4E) and AMD (FIGS. 4B, 4D, and 4F) RPE showing the disease phenotypes. Arrows indicate the observed morphological differences. Higher magnification insets show the observed phenotypes (in FIGS. 4B and 4D), in comparison with the normal cellular structures (in FIGS. 4C and 4E). L: Lysosomes; M: Mitochondria; RER: Rough ER; DM: disintegrated mitochondria; LD: Lipid droplets; GG: Glycogen granules; CF: cytoskeletal fascicles; AP: autophagosomes; MP: mitophagosomes.

FIGS. 5A-5F depict AMD iPSC-RPE exhibiting relevant cellular phenotypes. FIGS. 5A-5F show electron microscopy images of normal (FIG. 5A: 25R; FIG. 5C: 10R; FIG. 5E: 25R), AMD-RPE-iPSC-RPE (FIG. 5B: 32R; FIG. 5F: 9R) and AMD Skin iPSC-RPE (FIG. 5D: 005BF) from a dry AMD patient also show diseased phenotypes. Red arrows indicate the observed morphological differences. Higher magnification insets show disintegrated mitochondria (in FIG. 5B: 32R; and FIG. 5D: 005BF), in comparison with the normal mitochondria (in FIG. 5A: 25R). M: Mitochondria; DM: disintegrated mitochondria; LD: Lipid droplets; AP: autophagosomes. All scale bars represent 500 nm.

FIGS. 6A-6B depict AMD iPSC-RPE being more susceptible to oxidative stress and produce higher ROS. FIG. 6A shows cell viability assays of AMD and control iPSC-RPE treated with increasing concentrations of H₂O₂ for 48 hrs. Higher susceptibility of the AMD iPSC-RPE under oxidative stress conditions (0.1, 0.2 and 0.4 mM H₂O₂) is observed in AMD iPSC-RPE compared to normal iPSC-RPE. FIG. 6B shows ROS production under stress conditions is significantly higher in AMD iPSC-RPE. 005BF is generated from dry AMD patient skin cells.

FIGS. 7A-7D depict AMD iPSC-RPE expressing lower SOD2 defense, exhibit lower mitochondrial activity and present higher cytoplasmic glycogen concentration. FIG. 7A shows that AMD iPSC-RPE are not capable of increasing SOD2 expression under stress conditions. AMD and control iPSC-RPE were treated with 0.4 mM H₂O₂ for 2 hrs for 5 consecutive days after which RNA were extracted and analyzed via quantitative RT-PCR. As opposed to normal iPSC-RPE, the AMD iPSC-RPE are not responsive to stress conditions and do not increase the SOD2 gene expression under stress conditions. (p-value≤0.05). GAPDH was used to normalize the samples and the relative expression of each sample is compared. FIGS. 7B-7C show that AMD iPSC-RPE have significantly lower mitochondrial activity as compared to control iPSC-RPE, as indicated by ATP levels measured by a luminescence assay in the presence (FIG. 7B) and absence (FIG. 7C) of hexokinase inhibitor. FIG. 7D shows measurement of cytoplasmic glycogen accumulation by colorimetric assay showing higher concentration in AMD iPSC-RPE as compared to control iPSC-RPE. Asterisks (*) indicate statistical significance, determined by student t-test (p-value≤0.05). 005BF is from a dry AMD patient.

FIGS. 8A-8B depict analysis of autophagy dynamics in normal (FIG. 8A) and AMD (FIG. 8B) RPE. LC3 immunoblot of AMD and control RPE after starvation and in the presence or absence of IGF-1. Beta actin is used as a normalization control. FIGS. 8C-8D show ratios of the LC3-II/LC3-I levels as determined by densitometry illustrated in the graph, which shows that an increase in autophagy dynamics in the absence of IGF1 is observed only in normal, but not in AMD RPE. Densitometry was performed on three repeats of the experiment in (FIG. 8A) and standard deviations were calculated. Asterisks in FIG. 8C indicate significant differences of LC3-II/LC3-I ratios between samples treated and untreated with IGF1. Asterisks in FIG. 8D indicate significant differences of LC3-II/LC3-I ratios between non-starved and starved samples. FIG. 8E shows swollen LAMP-1-positive organelles, indicative of defective lysosomal function, are observed in AMD RPE (white arrowheads), but not in the normal RPE. Insets are 6× magnified boxed regions, scale bar represents 20 μm.

FIG. 9 depicts that the mTOR pathway is overactive in AMD. Normal (Ctrl 10) and AMD (AMD 14) RPE were starved in HBSS for 4 h followed by the addition of IGF1 for the indicated times. Over-activity of mTOR is shown by expression of its downstream target, the phosphorylated protein p70S6K, in the AMD RPE compared to control RPE.

FIG. 10A-10C depict that normal and AMD RPE were cultured in complete media. FIG. 10A shows that AKT3 gene expression is significantly lower in AMD RPE, whereas, AKT1 (FIG. 10B) and AKT2 (FIG. 10C) show similar expression pattern in AMD RPE and normal RPE. Asterisks (*) indicate p-values<0.05.

FIGS. 11A-11E depict AMD-iPSC-RPE that exhibit increased levels of PARP2 expression, and lower levels of SIRT-1 protein and PGC-1α expression as compared to normal iPSC-RPE. FIGS. 11A-11B show that the AMD RPE-iPSC-RPE generated from native AMD RPE (FIG. 11A) and the AMD Skin-iPSC-RPE (FIG. 11B) show higher levels of PARP2 expression as shown by quantitative Real Time PCR compared to normal RPE-iPSC-RPE. Graphs are represented as mean±S.E. of three independent experiments. Asterisk (*) shows statistically significance value analyzed by t-test as compared to 25R, p≤0.05. Hash tag (#) represents statistically significance value analyzed by t-test as compared to 10R, p≤0.05. FIG. 11C shows representative western blot image of three independent experiments showing lower SIRT-1 levels in AMD RPE-iPSC-RPE and AMD Skin-iPSC-RPE as compared to those in normal RPE-iPSC-RPE. FIG. 11D shows densitometry of average of three independent western blots analyses showing about a 2-fold decrease in SIRT-1 protein levels in AMD RPE-iPSC-RPE and AMD Skin-iPSC-RPE as compared to normal RPE-iPSC-RPE. FIG. 11E shows Quantitative Real Time PCR showing decreased PGC-1α expression in AMD-iPSC-RPE as compared to normal iPSC-RPE. Graph represents mean±S.E. of three independent experiments. Asterisk (*) shows statistically significance value analyzed by t-test as compared to 25R, p≤0.05. Hash tag (#) represents statistically significance value analyzed by t-test as compared to 10R, p≤0.05.

FIGS. 12A-12N depict human RPE cells (ARPE19) that were transfected with the empty pCMV6-Entry vector (FIGS. 12A-12C) or Flag-tagged CRM-1 in pCMV6-Entry (FIGS. 12D-12F) and stained with anti-Flag antibody at 48 hrs after the transfection to confirm the expression of Flag-CRM-1 in the nucleus of the transfected cells (FIGS. 12D-12F). The empty vector transfection (FIGS. 12A-12C) was used as negative control. FIGS. 12G-12H show phase images of the ARPE19 cells transfected with either the empty vector (FIG. 12G) or the Flag-tagged CRM-1 overexpressing plasmid (FIG. 12H) at 3 weeks after transfection and selection with G-418. The cells expressing exogenous CRM-1 (FIG. 12H) exhibit significant morphological differences, accumulating numerous vacuoles (red arrows) that were not found in the control (FIG. 12G). FIGS. 12I-12K show ARPE19 transfected with the empty vector, selected with G-418 and stained with anti-PGC-1α 3 weeks after transfection, show mainly nuclear localization of PGC-1α. FIGS. 12L-12N show ARPE19 transfected with the CRM-1 overexpressing plasmid, selected with G-418 and stained with anti-PGC-1α 3 weeks after transfection, show significantly lower levels of PGC-1α in the nucleus compared to the control cells in (FIGS. 12I-12K). Nuclei in FIGS. 12B, 12E, 12J, and 12M are stained with DAPI.

FIGS. 13A-13D depict electron microscopy micrographs of human RPE cells (ARPE19) transfected with the pCMV6-Entry empty vector (FIGS. 13A and 13B), or with the CRM-1 overexpressing plasmid (CRM-1 in pCMV6-Entry) (FIGS. 13C and 13D) at 3 weeks after transfection and selection with G-418. Red arrows indicate the observed ultrastructural differences between CRM-1 overexpressing and control cells. Control RPE cells (FIGS. 13A and 13B) have normal mitochondria (M), rough ER, higher cytoplasmic density and undergo normal mitosis as indicated by the presence of mitotic chromosomes (Ch) in (FIG. 13B). CRM-1 overexpressing RPE exhibit glycogen accumulation, with larger size glycogen granules (GG) in aggregates. Their cytoplasm is lighter in density compared to control RPE, with numerous vacuoles (V). Disintegrating mitochondria (DM), some forming concentric lamellar membranes (DM in FIG. 13C, top) that coalesce to form outer dense membrane of mitophagosomes (MP in FIG. 13D) are only seen in CRM-1 overexpressing RPE cells. Rough ER (RER) in the CRM-1 overexpressing RPE appears degenerated (in FIG. 13D), lacking ribosomes, as compared to control RPE (in FIG. 13A). Lysosomes (L) and autophagosomes (AP) are visible in the CRM-1 overexpressing RPE (FIGS. 13C-13D) and absent in control RPE (FIG. 13A-13B).

FIG. 14 depicts the hypothetic role of SIRT-1/PGC-1α repression in the pathophysiology of AMD. PARP2 can down regulate SIRT-1 function by binding to the SIRT-1 promoter, inhibiting transcription and resulting in reduced SIRT-1 levels. The reduction in SIRT-1 activity would reduce deacetylation rate of PGC-1α. The resulting PGC-1α hyperacetylation decreases its activity which translates to lower mitochondrial content and activity, lowered mitochondrial respiratory capacity, lowered ROS detoxification and increased ROS production, contributing to the pathophysiology of AMD.

FIGS. 15A-15B depict a normal and (FIG. 15B) retina and a retina from a subject suffering from dry AMD (FIG. 15A). The retina from the subject with dry AMD show drusen as spots (FIG. 15A).

FIGS. 16A-16G depict the effects of telmisartan on PGC-1α gene expression, cell viability and AMPK phosphorylation in normal (control) and AMD retinal pigment epithelium. FIGS. 16A, 16B and 16C show telmisartan increasing PGC-1α and Acyl-CoA Dehydrogenase (ACADM) but not PGC-1β expression in normal (control) and AMD retinal pigment epithelium (RPE). FIG. 16D shows telmisartan inducing AMPK phosphorylation in normal (control) and AMD RPE. FIGS. 16E, 16F and 16G show telmisartan and PQQ increasing cell viability of AMD RPE under oxidative stress.

FIGS. 17A-17H show EM of eye sections of PGC-1α mice fed with HFD exhibiting severe abnormalities in RPE. FIG. 17A shows section of WT mouse eye fed with RD showing RPE normal phenotype. FIG. 17B shows higher magnification of FIG. 17A. FIG. 17C shows eye section of WT mouse fed with HFD. FIG. 17D shows higher magnification of FIG. 17C, showing damaged mitochondria (flat arrow), lipid droplets (arrowhead) and lipofuscin (concave arrow) in RPE. FIGS. 17E and 17F show eye section of PGC-1α^{+/-} mouse fed with RD showing presence of lipid droplets (arrowhead), lipofuscin (concave arrow) and mitochondrial damage (flat arrow) in RPE. FIGS. 17G and 17H show eye section of PGC-1α^{+/-} mouse fed with HFD demonstrating the presence of mitochondrial damage (flat arrow), numerous lipid droplets (arrowhead) and lipofuscin (concave arrow). Ten different pictures of each sample were randomly selected lipid droplets were counted in WT and PGC-1α^{+/-} under RD and HFD diet. All counted images were the same size and the same magnification (8000×). Ratio of lipid: PGC-1α^{+/-}(RD)/WT (RD)=16; PGC-1α^{+/-}(HFD)/WT (HFD)*=1.5.

FIGS. 18A-18B depict the retina of the PGC-1α^{+/-} mice exhibit higher inflammatory response to peritoneal LPS injection. WT (3) and PGC-1α^{+/-} (3) mice were injected with LPS (0.5 mg/kg, i.p.). 24 hours after injection mice were sacrificed in 20-minute intervals. Eyes were enucleated and retina extracted for RNA isolation followed by Real Time PCR for TNFα (FIG. 18A) and INFγ (FIG. 18B).

FIGS. 19A-19E depict gene expression profile of various genes associated with AMD in heterologous mice on a high fat diet (HFD) and wild-type (WT) mice.

FIGS. 20A-20C depict effects of telmisartan (FIG. 20A), rucaparib (UPF 1069) (FIG. 20B) and fenofibrate (FIG. 20C) incorporated in high fat dieat on regulating the AMD-like gene expression, APOB, in WT and PGC-1α+/−(HET) mice. HFD increases the APOB expression in the retina of the HET mice. UPF 1069 and fenofibrate significantly reduced the APOB levels in the retina of WT. All three compounds were able to significantly reduce the APOB levels in the retina of the HET mice.

FIGS. 21A-21C depict effects of telmisartan (FIG. 21A), rucaparib (UPF 1069) (FIG. 21B) and fenofibrate (FIG. 21C) incorporated in high fat dieat on regulating the AMD-like gene expression, APP, in WT and PGC-1α+/−(HET). Only rucaparib (UPF 1069) was able to significantly reduce the APP levels in the retina of the HET mice. Telmisartan and Fenofibrate even induced a significant increase in the APP levels in the retina of the WT mice and were unable to decrease the APP levels in the HET mice.

FIGS. 22A-22C depict effects of telmisartan (FIG. 22A), rucaparib (UPF 1069) (FIG. 22B) and fenofibrate (FIG. 22C) incorporated in high fat dieat on regulating the AMD-like gene expression, APOE, in WT and PGC-1α+/−(HET). All three compounds are able to significantly reduce the APOE levels in the retina of the WT and HET mice.

FIGS. 23A-23C depict effects of telmisartan (FIG. 23A), rucaparib (UPF 1069) (FIG. 23B) and fenofibrate (FIG. 23C) incorporated in high fat diet on regulating the AMD-like gene expression, APOJ, in WT and PGC-1α+/−(HET). UPF 1069 and fenofibrate significantly reduced the APOJ expression in the retina of HET mice. Fenofibrate significantly increased the APOJ levels in the retina of the WT mice. Telmisartan did not reduce the APOJ levels.

FIGS. 24A-24C depict effects of telmisartan (FIG. 24A), rucaparib (UPF 1069) (FIG. 24B) and fenofibrate (FIG. 24C) incorporated in high fat diet on regulating PGC-1α gene expression in WT and HET mice. All three compounds were able to significantly Increase PGC-1α expression in HET mice. Telmisartan and rucaparib also significantly increased the PGC-1α expression in WT mice.

### DETAILED DESCRIPTION OF THE INVENTION

The present invention is related to diagnostic, treatment and compound screening methods related to dry age-related macular degeneration (dry AMD) and/or drusen-related genes. In some embodiments, the methods comprising determining expression or activity levels of peroxisome proliferator-activated receptor-gamma coactivator 1-alpha (PGC-1α) in a sample obtained from the subject, and comparing the determined expression or activity levels of PGC-1α in the subject with the expression or activity levels obtained from subjects determined to define normal expression or activity levels of PGC-1α to determine if the subject's expression or activity levels of PGC-1α are altered compared to normal expression or activity levels of PGC-1α. A reduction in the expression or activity levels of PGC-1α in the sample compared to normal levels is indicative that the subject is at risk of developing dry AMD.

In other embodiments, the methods comprise determining the levels of acetylated PGC-1α in a sample obtained from the subject, and comparing the determined levels of acetylated PGC-1α in the subject with the levels of acetylated PGC-1α obtained from subjects determined to define normal levels of acetylated PGC-1α to determine if the subject's levels of acetylated PGC-1α are altered compared to normal levels of acetylated PGC-1α. An increase in the levels of acetylted PGC-1α in the sample compared to normal levels is indicative that the subject is at risk of developing dry AMD.

In other embodiments, the methods comprise determining mRNA levels of RAC-gamma serine/threonine-protein kinase (AKT3) in a sample obtained from the subject, and comparing the determined mRNA levels of AKT3 in the subject with the mRNA levels obtained from subjects determined to define normal mRNA levels of AKT3 to determine if the subject's mRNA levels of AKT3 are altered compared to normal mRNA levels of AKT3. A reduction in the mRNA levels of AKT3 in the sample compared to normal mRNA levels is indicative that the subject is at risk of developing dry AMD.

In other embodiments, the methods comprise determining expression or activity levels of poly(adenosine diphosphate ribose) polymerase-2 (PARP2) in a sample obtained from the subject, and comparing the determined expression or activity levels of PARP2 in the subject with the expression or activity levels obtained from subjects determined to define normal expression or activity levels of PARP2 to determine if the subject's expression or activity levels of PARP2 are altered compared to normal expression or activity levels of PARP2. An increase in the expression or activity levels of PARP2 in the sample compared to normal levels is indicative that the subject is at risk of developing dry AMD.

In other embodiments, the methods comprise determining expression or activity levels of NAD-dependent deacetylase sirtuin-1 (SIRT-1) in a sample obtained from the subject, and comparing the determined expression or activity levels of SIRT-1 in the subject with the expression or activity levels obtained from subjects determined to define normal expression or activity levels of SIRT-1 to determine if the subject's expression or activity levels of SIRT-1 are altered compared to normal expression or activity levels of SIRT-1. A reduction in the expression or activity levels of SIRT-1 in the sample compared to normal levels is indicative that the subject is at risk of developing dry AMD.

In other embodiments, the methods comprise determining expression or activity levels of AMP-activated protein kinase (AMPK) in a sample obtained from the subject, and comparing the determined expression or activity levels of AMPK in the subject with the expression or activity levels obtained from subjects determined to define normal expression or activity levels of AMPK to determine if the subject's expression or activity levels of AMPK are altered compared to normal expression or activity levels of AMPK. A reduction in the expression or activity levels of AMPK in the sample compared to normal levels is indicative that the subject is at risk of developing dry AMD.

Age-related macular degeneration is a progressive disease that can lead to permanent loss of vision. AMD is distinguished from acute retinal damage in that the disease is age-related and generally starts with accumulation of drusen underneath the RPE, progressively causing RPE dysfunction and ultimately leading to photoreceptor loss. AMD can, but not always, advance very slowly and vision loss may not occur for a long time. In others, AMD can progress faster and may lead to a loss of vision in one or both eyes. AMD differs from acute retinal damage induced either by injuries or sun damage, neither of which are caused by aging or marked by the presence of drusen.

In one embodiment, the AMD that is treated, diagnosed or subject to the methods of the present invention is dry AMD. In another embodiment, the AMD that is treated, diagnosed or subject to the methods of the present invention is wet AMD. In another embodiment, the AMD that is treated, diagnosed or subject to the methods of the present invention contain at least one hallmark of dry AMD, such as but not limited to activity or expression levels of drusen-related genes. Thus, the methods of the present invention can be practiced on subjects that have symptoms or markers of both dry and wet AMD.

The term “dry AMD” is well known in the art and is used to mean the condition of age-related macular degeneration marked by the presence of drusen, alterations in retinal pigment epithelium (RPE), accumulation of immune cells such as macrophages and microglia, thickening of Bruch's membrane (including excessive cholesterol and calcium accumulation therein), general atrophy, alterations in the choriocapillaris, degeneration of photoreceptors, and cell death. The appearance of drusen is generally considered one of the first detectable symptoms of AMD, in particualr dry AMD. One of skill in the art would understand and be able to readily identify drusen in a subject or a tissue sample taken from a subject. Drusen are deposits that typically comprise acute phase proteins, such as but not limited to, C-reactive protein, vitronectin, a-antichymotrypsin, amyloid P component, and fibrinogen, as well as complement pathway components, such as but not limited to C3, C5 and C5b-9 complex as well as apolipoproteins B and E, mucopolysaccarides, lipids, mannose, crystallins, immunoglobulins, and sialic acid.

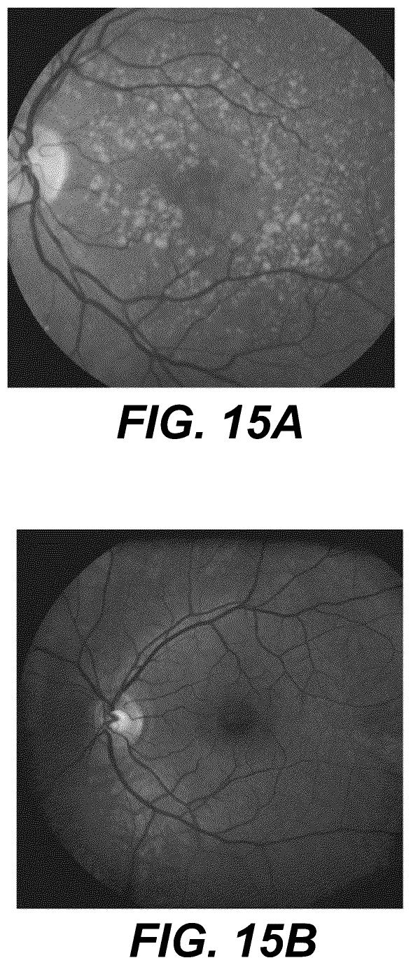

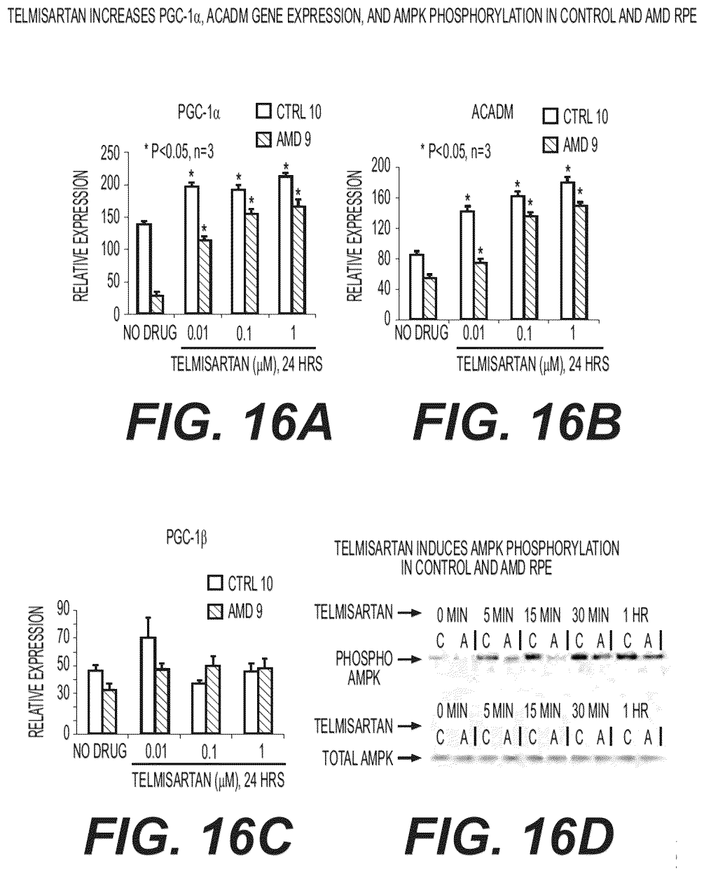

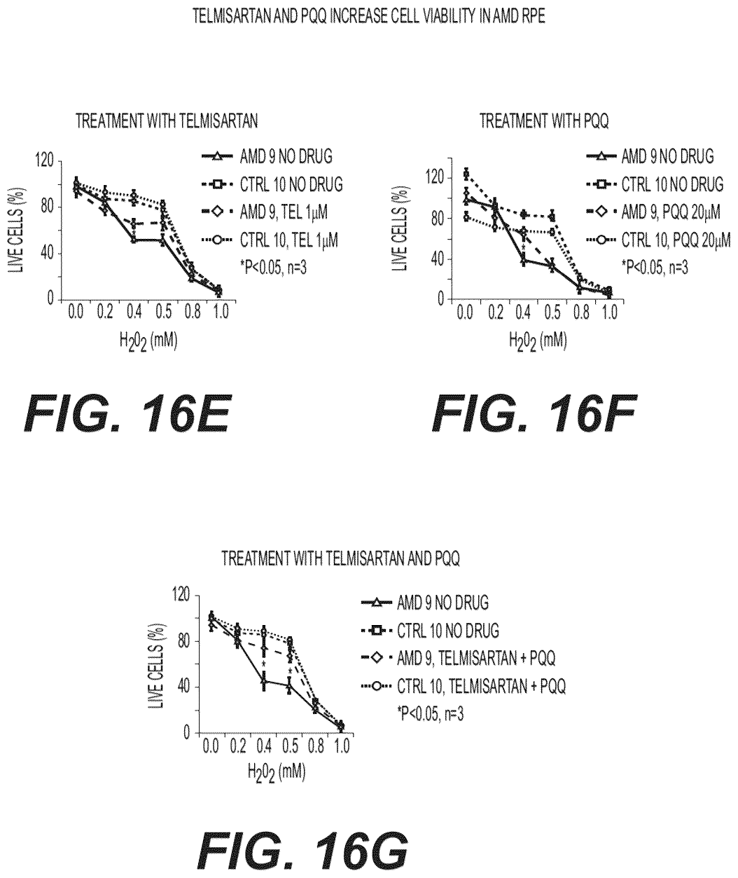

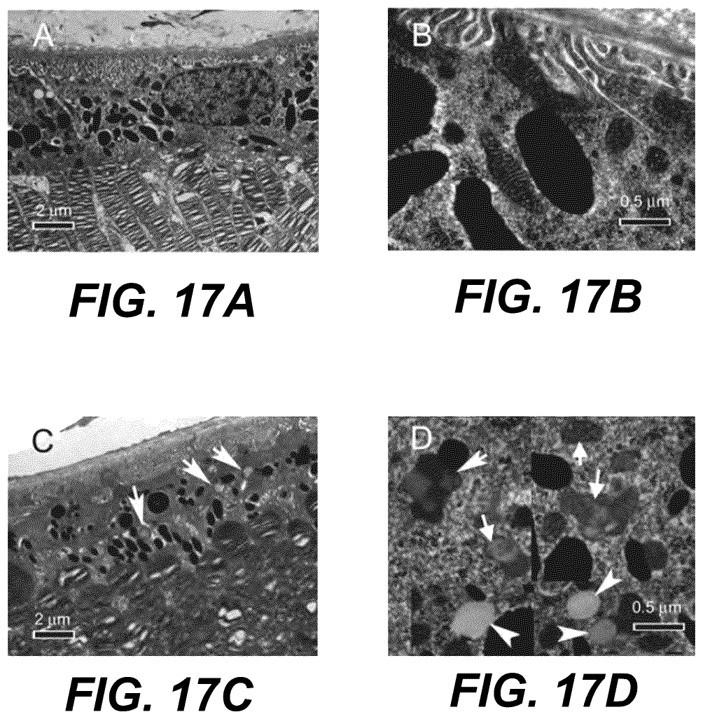

As shown in FIG. 15, dry AMD is marked by the presence of drusen and vision loss in dry AMD generally occurs through loss of photoreceptors. In general, dry AMD is also considered the non-neovascular AMD, but, as used herein, dry AMD can include some neovascularization, provided that the subject has at least one hallmark of dry AMD. In other embodiments, the subject with dry AMD is that treated, diagnosed or subjected to the methods of the present invention has no detectable hallmarks of wet AMD.

Wet AMD, on the other hand, is also well known and is marked by abnormal blood vessel growth in the macula. Ultimately, bleeding and protein leakage can occur through these newly formed blood vessels, which causes loss or photoreceptors and subsequent vision damage. Wet AMD almost always begins with dry AMD, although not all instances of dry AMD will progress to wet AMD. In select embodiments, the methods of the present invention are related to the diagnosis, treatment and methods of screening compounds useful for treating AMD marked by the presence of drusen without corresponding neovascularization of the macula, i.e., dry AMD. In other embodiments, the methods of the present invention are related to the diagnosis, treatment and methods of screening compounds useful for treating AMD marked by the presence of drusen with corresponding neovascularization of the macula, i.e., wet AMD.

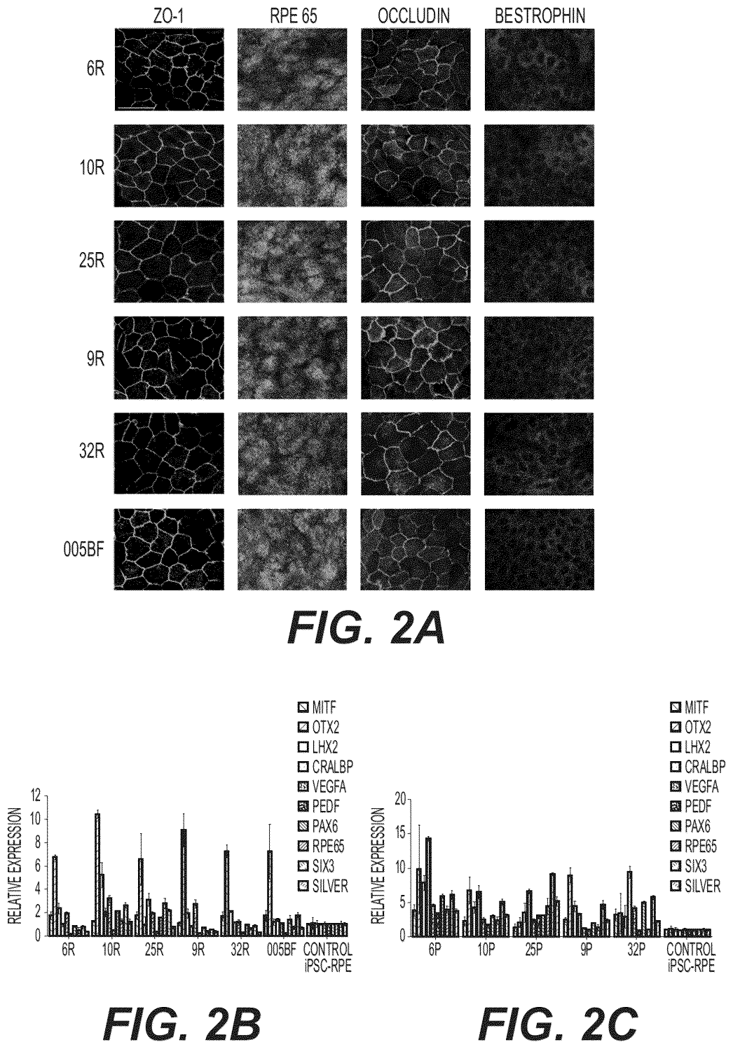

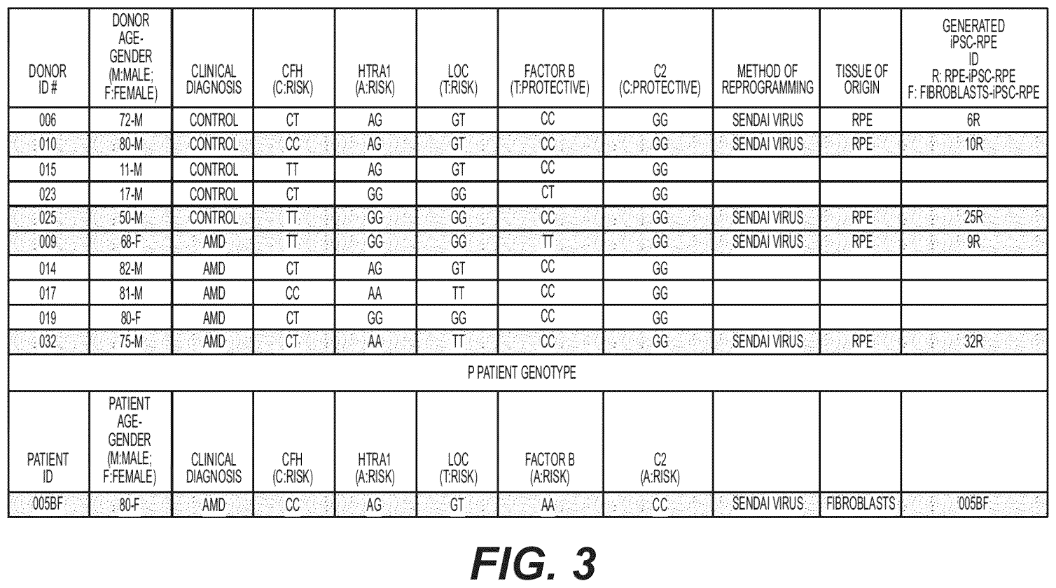

For example, Egger, A. et al., PLoS ONE, 7(2): e31272 (2012) (doi:10.1371/journal.pone.0031272), which is incorporated by reference, shows morphological sections of retinas from normal and knock-out mice that were not stressed with exposure to white light (2A) and morphological sections of retinas from normal and knock-out mice that were stressed by exposure to white light (3A). FIG. 3A shows the absence of drusen, and shows acute retinal damage caused by exposure to white light (after pupil dilation). In contrast, FIG. 2 of Spaide, R and Curcio, C., Retina, 30(9): 1441-1454 (2010), shows a retinal section from a subject in which the macula contains drusen. The conditions on which the methods of the present invention are distinguished from the condition shown in Egger at al. in that the conditions on which the methods of the present invention are practices are generally marked by the presence of drusen and are not conditions caused by acute retinal damage.

As used herein, the term subject or “test subject” indicates a mammal, in particular a human or non-human primate. The test subject may or may not be in need of an assessment of a predisposition of dry AMD. For example, the test subject may have a condition or may have been exposed to injuries or conditions that are associated with AMD prior to applying the methods of the present invention. In another embodiment, the test subject has not been identified as a subject that may have a condition or may have been exposed to injuries or conditions that are associated with dry AMD prior to applying the methods of the present invention.

As used herein, the term means “increased risk” is used to mean that the test subject has an increased chance of developing or acquiring dry AMD to a normal individual. The increased risk may be relative or absolute and may be expressed qualitatively or quantitatively. For example, an increased risk may be expressed as simply determining the subject's levels of PGC-1α, acetylated PGC-1α, AKT3, PARP2 and/or SIRT-1 and placing the patient in an “increased risk” category, based upon previous population studies. Alternatively, a numerical expression of the subject's increased risk may be determined based upon the expression or activity levels. As used herein, examples of expressions of an increased risk include but are not limited to, odds, probability, odds ratio, p-values, attributable risk, relative frequency, positive predictive value, negative predictive value, and relative risk.

For example, the correlation between a subject's expression or activity levels and the likelihood of suffering from dry AMD may be measured by an odds ratio (OR) and by the relative risk (RR). If P(R⁺) is the probability of developing AMD for individuals with the risk profile (R) and P(R⁻) is the probability of developing AMD for individuals without the risk profile, then the relative risk is the ratio of the two probabilities: RR=P(R⁺)/P(R⁻).

In case-control studies, however, direct measures of the relative risk often cannot be obtained because of sampling design. The odds ratio allows for an approximation of the relative risk for low-incidence diseases and can be calculated: OR=(F⁺/(1−F⁺))/(F⁻/(1−F⁻)), where F⁺ is the frequency of expression or activity levels in cases studies and F⁻ is the frequency of expression or activity levels or mRNA levels in risk profile of controls. F⁺ and F⁻ can be calculated using the frequencies of expression or activity levels of the study.

The attributable risk (AR) can also be used to express an increased risk. The AR describes the proportion of individuals in a population exhibiting dry AMD. AR may also be important in quantifying the role of individual factors in disease etiology and in terms of the public health impact of the individual marker. The public health relevance of the AR measurement lies in estimating the proportion of cases of dry AMD in the population that could be prevented if the factors were absent. AR may be determined as follows: AR=P_{E}(RR−1)/(P_{E}(RR−1)+1), where AR is the risk attributable to a expression level or mRNA level or individual factors of the profile, and P_{E} is the frequency of the expression level or mRNA level or individual factors of the profile within the population at large. RR is the relative risk, which can be approximated with the odds ratio when the expression level or mRNA level or individual factors of the profile under study has a relatively low incidence in the general population.

In one embodiment, the increased risk of a patient can be determined from p-values that are derived from association studies. Specifically, associations with a specific expression level or mRNA level can be performed using regression analysis by regressing the expression level or mRNA level with dry AMD. In addition, the regression may or may not be corrected or adjusted for one or more factors. The factors for which the analyses may be adjusted include, but are not limited to age, sex, weight, ethnicity, geographic location, fasting state, state of pregnancy or post-pregnancy, menstrual cycle, general health of the subject, alcohol or drug consumption, caffeine or nicotine intake and circadian rhythms, to name a few.

Increased risk can also be determined from p-values that are derived using logistic regression. Binomial (or binary) logistic regression is a form of regression that is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring. These analyses can be conducted with the program SAS.

SAS (“statistical analysis software”) is a general-purpose package (similar to Stata and SPSS) created by Jim Goodnight and N.C. State University colleagues. Ready-to-use procedures handle a wide range of statistical analyses, including but not limited to, analysis of variance, regression, categorical data analysis, multivariate analysis, survival analysis, psychometric analysis, cluster analysis, and nonparametric analysis.

The sample that is subjected to the methods of the present invention may or may not be processed prior assaying expression or activity levels of the various markers of dry AMD. For example, whole blood may be taken from an individual and the blood sample may be processed, e.g., centrifuged, to isolate plasma or serum from the blood. The sample may or may not be stored, e.g., frozen, prior to processing or analysis. In one embodiment, the sample on which the methods of the present invention are performed is taken from the subject's blood. In one embodiment, the test sample is whole blood. In another embodiment, the test sample is serum. In another embodiment, the test sample is plasma.

In another embodiment, the sample on which the methods of the present invention are performed is a cell sample. In one specific aspect, the cell sample is a sample of RPE cells. As used herein, retinal pigment epithelial (RPE) cells are polarized epithelial cells that exhibit both phenotypic and functional characteristics that are common and well-known to native RPE cells. The RPE cells need not exhibit every single characteristic of native RPE cells, but the characteristics of the RPE cells used in the methods of the invention herein should be consistent with characteristics of native RPE cells. As used herein, “native RPE cells” are cells that have not been recombinantly manipulated in any way and naturally exhibit the phenotypic and functional characteristics of RPE cells. Native RPE cells can be found in in vivo and in vitro environments. In one embodiment, the RPE cells used in the methods of the present invention are native RPE cells.

In another embodiment, the RPE cells used in the methods of the present invention are not native RPE cells, but are generated RPE cells. For example, non-native RPE cells, such as but not limited to fibroblasts, may be obtained from a subject and stem cells may be generated using well-known methods for generating induced pluripotent stem cells (iPSCs). These iPSCs can, in turn, be used to generate RPE cells (iPSC-RPE). Techniques for generating iPSCs as well as generating RPE from iPSCs are known in the art. See, e.g., Kamao, H., et al., Stem Cell Reports, 2(2):205-218 (2014), and Kokkinaki, M., et al., Stem Cells, 29(5):825-835 (2011), which are incorporated by reference. These iPSC-RPE cells can then be used in the methods of the present invention. Accordingly the term “sample” as used herein can mean a sample of cells taken from a subject, with the cell sample being used to subsequently generate iPSC-RPE.

As used herein, generated functional RPE cells are cells that are initially non-RPE cells and are subsequently transformed into functional RPE cells. The term “transformed,” when used in connection with generating functional RPE cells, is not limited to transfection and other genetic recombinant techniques. For example, it may be possible to isolate non-RPE cells and submit the cells to culture conditions that allow the cells to de-differentiate into a stem-cell like cell, and subsequently alter the culture conditions to drive the cells' towards a phenotype of functional RPE cells.

Of course, generation of non-RPE cells into functional RPE cells can include transfection or other genetic recombinant techniques. In select embodiments, the non-RPE cells that are isolated and used in the methods of the present invention are first transformed into functional RPE cells using methods and techniques described in pending U.S. application Ser. No. 14/211,515, which is incorporated by reference.

Phenotypic and functional characteristics of native RPE cells include but are not limited to, presence or expression of melanin, presence or expression of pigment epithelium-derived factor (PEDF), presence of expression of RPE65, presence or expression of cellular retinaldehyde binding protein (CRALBP), presence or expression of bestrophin, presence or expression of Pax6 (although Pax6 is normally downregulated mature RPE cells), in the Na+/K+-ATPase being localized apically in the plasma membrane, the extracellular matrix metalloproteinase inducer (EMMPRIN) being located apically, N-CAM being located apically, αvβ5 integrin being located apically, chloride-bicarbonate exchange transporter being located basolaterally, Ca+-sensitive chloride channels being located basolaterally, syntaxin 2 (isoforms 2A and 2B) being located basolaterally, reduction or absence of syntaxin 3 expression, presence or expression of orthodentical homeobox 2 (OTX2), presence or expression of LIM homeobox 2 (LHX2), presence or expression of ectonucleoside triphosphate diphosphohydrolase 2 (ENTPD2), polarized secretion of vascular endothelial growth factor (VEGF), ability to form and maintain tight junctions, presence of a transepithelial potential (TEP), ability to perform phagocytosis, ability to form a confluent monolayer in culture, to name a few. Other characteristics of RPE cells include, but are not limited to those characteristics discussed in Kokkinaki, M., et al., Stem Cells, 29:825-835 (2011).

As used herein “non-RPE cells” are cells that do not have all three characteristics of the ability to perform phagocytosis, expression of melanin and expression of RPE65. It is, however, possible that the non-RPE cells used in the methods of the present invention may exhibit one or more phenotypic or functional characteristics of native RPE cells. In one embodiment, the non-RPE cells used in the methods of the present invention do not express RPE65. In another embodiment, the non-RPE cells used in the methods of the present invention do not express melanin. In another embodiment, the non-RPE cells used in the methods of the present invention do not express melanin and do not express RPE65. In yet another embodiment, the non-RPE cells used in the methods of the present invention do not have the ability to perform phagocytosis, do not express melanin and do not express RPE65.

In one embodiment, the non-RPE cells used in the methods of the present invention are neither embryonic stem cells, nor are they induced pluripotent stem cells (iPSCs). In another embodiment, the non-RPE cells are not adult stem cells. In another embodiment, the non-RPE cells are mesenchymal stem cells. In another embodiment, the non-RPE cells are blood cells, fibroblasts or epithelial cells. The non-RPE fibroblasts used in the methods of the present invention can be derived from any connective tissue, including but not limited to, dermis, adipose, bone and cartilage. In one specific embodiment, the non-RPE fibroblasts cells are dermal fibroblasts. In another embodiment, the non-RPE cells are epithelial cells. The non-RPE epithelial cells used in the methods of the present invention can be derived from any epithelial tissue and/or blood cells including, but not limited to, digestive system epithelium, skin epithelium, respiratory system epithelium, reproductive system epithelium and urinary system epithelium to name a few.

In select embodiments, the methods comprise generating functional RPE from non-RPE cells. Methods of generating functinal RPE cells from non-RPE cells may comprise administering at least one gene or gene product to non-RPE cells in an amount sufficient to transform the non-RPE cells into functional RPE cells, wherein the at least one gene or gene product is selected from the group consisting of Pax6, OTX2, LHX2, Six3, Six6, Sox9, Nr2f2, ENTPD2, ELF3 and MITF. Accordingly the term “sample” as used herein can mean a sample of non-RPE cells taken from a subject, with the cell sample being used to subsequently generate functional RPE cells.

In some embodiments, the methods of the present invention can be performed in culture. When performed in culture, standard, well-known methods for culturing non-RPE can be used. In one specific embodiment, the non-RPE cells are initially seeded onto cell culture surfaces without any matrix or cellular scaffold being present. In another embodiment, the non-RPE cells are initially seeded onto cell culture surfaces with a matrix or cellular scaffold being present. Cellular scaffold and matrices for culturing RPE and non-RPE cells are well known in the art. For example, Thompson, H. A., et al., J. Biomed. Mat. Res. A, 95A(4):1233-1243 (2010) and Lu, L., et al., Biomaterials, 22:3345-3355 (2001), both of which are incorporated by reference, disclose matrices upon which RPE cells can be cultured. Other cell culture matrices include, but are not limited to MATRIGEL™, collagen, laminin, fibronectin and the like.

The methods may also include administering any combination of one or more genes or gene products to produce functional RPE cells. For example, genes or gene products of each of Pax6, OTX2, LHX2, Six3, Six6, Sox9, Nrf2f, ENTPD2, ELF3 and MITF can be administered alone to the non-RPE cells. In another embodiment, genes or gene products of Nrf2f and ENTPD2 can be administered to the non-RPE cells. In another embodiment, genes or gene products of Nrf2f, ENTPD2 and ELF3 can be administered to the non-RPE cells. In another embodiment, genes or gene products of Nrf2f, ENTPD2, ELF3 and MITF can be administered to the non-RPE cells. In another embodiment, genes or gene products of Nrf2f and ELF3 can be administered to the non-RPE cells. In another embodiment, genes or gene products of Nrf2f, ELF3 and MITF can be administered to the non-RPE cells. In another embodiment, genes or gene products of Nrf2f and MITF can be administered to the non-RPE cells. In another embodiment, genes or gene products of Nrf2f, ENTPD2 and MITF can be administered to the non-RPE cells. In another embodiment, genes or gene products of ENTPD2 and ELF3 can be administered to the non-RPE cells. In another embodiment, genes or gene products of ENTPD2 and MITF can be administered to the non-RPE cells. In another embodiment, genes or gene products of ENTPD2, ELF3 and MITF can be administered to the non-RPE cells. In another embodiment, genes or gene products of ELF3 and MITF can be administered to the non-RPE cells.

The source of the non-RPE cells, if used, can be any animal source; for example the source of the non-RPE cells can be human, non-human primate, canine, porcine, feline, bovine, equine rodent. In general, the source of the cells, i.e., human, mouse, etc., would determine the source of the genes or gene products that are administered to the non-RPE cells. For example, a human gene encoding ENTPD2 would be administered to human non-RPE cells. This matching of the source gene or gene product with the source cells, however, is not necessary. For example, a mouse ENTPD2 gene or gene product may be administered to non-RPE cells derived from a source other than a mouse, e.g. a rat or human. If more than one gene or gene product is administered to the non-RPE cells, the administration of the genes or gene product can be sequentially or concurrently, and not all the genes or gene products need be from the same animal source.

The invention is not limited to the quality or quantity of characteristics of functional RPE cells used in the methods of the present invention. The term “functional RPE cells,” means the cells at least have the ability to perform phagocytosis, express melanin and express RPE65. Of course, functional RPE cells can possess additional characteristics consistent with native RPE cells, such as those discussed in Kokkinaki, M., et al., Stem Cells, 29:825-835 (2011).

As used herein, the “presence or expression” of a particular protein or marker can be assessed by detecting or determining the protein levels. The “presence or expression” of a particular protein or marker can also be assessed by detecting or determining the by detecting or determining the mRNA levels that correspond to the gene or marker being expression. Thus “expression levels” can mean protein levels or mRNA levels for a given gene or marker. Alternatively, the “presence or expression” of a particular protein or marker can also be assessed with functional assays, whereby the cell is able to perform a specific function based on the presence of a functional protein or marker, for example, measuring a product or by-product from a chemical reaction in which the marker or protein being assayed takes part. Other functional assays that might be used to assess the presence or expression of a particular protein or marker might include measuring transmembrane standing potential at various places, e.g., apical or basal ends, in the cell. The invention is not limited by the methods of determining the presence or expression of a particular protein or marker. One of skill in the art can readily understand and appreciate numerous methods to determine the presence or expression (or absence thereof) of a particular marker or protein.

Methods of assessing functional characteristics of cells, for example to determine if cells are functional RPE cells, are well known in the art. For example, in vitro assays utilizing latex beads can be used to assess the ability of cells to perform phagocytosis. See Kilmanskaya, I., Meth. Enzymol., 418:169-194 (2006), which is incorporated by reference. Other in vitro phagocytosis assays include, but are not limited to, phagocytosis assays utilizing rod outer segments as described in Finnemann, S., et al., Proc. Nat'l. Acad. Sci., 94(24):12932-12937 (1997), which is incorporated by reference. In addition, polarity assays, for example to if the Na+/K+ ATPase is located on the apical portion of the plasma membrane, are well known in the art and are discussed in Kokkinaki, M., et al., Stem Cells, 29:825-835 (2011), which is incorporated by reference.

Techniques to assay expression or activity levels from test samples are well known to the skilled technician, and the invention is not limited by the means by which the components are assessed. In one embodiment, expression or activity levels of PGC-1α, acetylated PGC-1α, AKT3, PARP2 and/or SIRT-1 (phosphorylation), AMPK (phosphorylation) are assessed using, PCR, quantitative PCR, Western blot, Immunoprecipitation (IP), Northern blot, Southern blot, ELISA assays, mass spectrometry in conjunction with ultra-performance liquid chromatography (MS-UPLC), high-performance liquid chromatography (HPLC), and UPLC to name a few. The methods of assessing the expression or activity levels of PGC-1α, acetylated PGC-1α, AKT3, PARP2 and/or SIRT-1 will depend on the type of molecule to be assessed, e.g., protein expression levels or mRNA levels may or may not be assessed by different methods than for assessing mRNA levels. For example, AMPK can phosphorylate PGC-1α at threonine 177 and serine-538 to activate PGC-1α, thus phosphorylation levels of PGC1α at these sites can be used as an assessment of PGC-1α activity levels.

The assessment of expression or activity levels of PGC-1α, acetylated PGC-1α, AKT3, PARP2 and/or SIRT-1 levels can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing.

In one embodiment, each of the methods of the present invention herein comprise measuring more than one of expression or activity levels of PGC-1α, acetylated PGC-1α, AKT3, PARP2 and/or SIRT-1. In one specific embodiment, the methods of the present invention herein comprise first measuring expression or activity levels of at least one of PGC-1α, acetylated PGC-1α, AKT3, PARP2 and/or SIRT-1, followed by measuring expression or activity levels of another of PGC-1α, acetylated PGC-1α, AKT3, PARP2 and/or SIRT-1. Any combination of two or more markers in any order can be used in any of the methods of the present invention. In these embodiments, the results from an analysis of the first component can be used as a screening tool for further analysis of the second and subsequent component(s). For example, the expression or activity levels of PARP2 are assessed for a subject and, based on these initial results, a sample taken from the subject can then be assayed for levels of acetylated PGC-1α, the combination of which can then be used to determine the subject's likelihood or risk of suffering from dry AMD.

The subject's expression or activity levels of PGC-1α, acetylated PGC-1α, AKT3, PARP2 and/or SIRT-1 are compared to the expression or activity levels of PGC-1α, acetylated PGC-1α, AKT3, PARP2 and/or SIRT-1, respectively, that are deemed to be a normal levels. To establish the levels of a normal individual, an individual or group of individuals may be first assessed symptoms or signs of dry AMD to establish that the individual or group of individuals has normal, healthy or acceptable eyes. Once established, the expression or activity levels of PGC-1α, acetylated PGC-1α, AKT3, PARP2 and/or SIRT-1 of the individual or group of individuals can then be determined to establish “normal levels.” In one embodiment, normal expression or activity levels of PGC-1α, acetylated PGC-1α, AKT3, PARP2 and/or SIRT-1 can be ascertained from the same subject when the subject is deemed to possess normal or healthy vision with no detectable signs of (clinical or otherwise) of dry AMD. In one embodiment, “normal levels” are assessed in the same subject from whom the sample is taken prior to the onset of measureable, perceivable or diagnosed dry AMD. That is, the term “normal levels” with respect to expression or activity levels of PGC-1α, acetylated PGC-1α, AKT3, PARP2and/or SIRT-1 can be used to mean the subject's baseline levels prior to the onset of dry AMD. The expression or activity levels of PGC-1α, acetylated PGC-1α, AKT3, PARP2 and/or SIRT-1 can then be reassessed periodically and compared to the subject's baseline levels.

Thus, the present invention also include methods of monitoring the progression of dry AMD in a subject, with the methods comprising determining the subject's expression or activity levels of PGC-1α, acetylated PGC-1α, AKT3, PARP2 and/or SIRT-1 more than once over a period of time. For example, some embodiments of the methods of the present invention will comprise determining the subject's expression or activity levels of PGC-1α, acetylated PGC-1α, AKT3, PARP2 and/or SIRT-1 two, three, four, five, six, seven, eight, nine, 10 or even more times over a period of time, such as a year, two years, three, years, four years, five years, six years, seven years, eight years, nine years or even 10 years or longer. The methods of monitoring a subject's risk of suffering from dry AMD would also include embodiments in which the subject's levels are assessed during and after treatment of dry AMD. In other words, the present invention also includes methods of monitoring the efficacy of treatment of dry AMD by assessing the subject's expression or activity levels of PGC-1α, acetylated PGC-1α, AKT3, PARP2 and/or SIRT-1 over the course of the treatment and after the treatment. The treatment may be any treatment designed to slow the progression or reverse the symptoms or causes of dry AMD, i.e., improve or slow the digression of a subject's vision.

In another embodiment, normal expression or activity levels of PGC-1α, acetylated PGC-1α, AKT3, PARP2 and/or SIRT-1 are assessed in a sample from a different subject or patient (from the subject being analyzed) and this different subject does not have or is not suspected of having dry AMD. In still another embodiment, the normal levels are assessed in a population of healthy individuals, the constituents of which display no signs of dry AMD. Thus, the subject's expression or activity levels of PGC-1α, acetylated PGC-1α, AKT3, PARP2 and/or SIRT-1 can be compared to normal levels generated from a single normal sample or levels generated from more than one normal sample.

Of course, measurements of PGC-1α, acetylated PGC-1α, AKT3, PARP2 and/or SIRT-1, e.g., concentration, of the normal expression or activity levels of PGC-1α, acetylated PGC-1α, AKT3, PARP2 and/or SIRT-1 can fall within a range of values, and values that do not fall within this “normal range” are said to be outside the normal range. These measurements may or may not be converted to a value, number, factor or score as compared to measurements in the “normal range.” For example, a measurement for expression or activity levels of PARP2 that are below the normal range, may be assigned a value or −1, −2, −3, etc., depending on the scoring system devised.

In select embodiments, expression or activity levels of PGC-1α, acetylated PGC-1α, AKT3, PARP2 and/or SIRT-1 need not be statistically significant for there to be considered a change from normal levels. In other embodiments, expression or activity levels of PGC-1α, acetylated PGC-1α, AKT3, PARP2 and/or SIRT-1 can be statistically significant from normal levels.

If it is determined that a subject has an increased risk of suffering from dry AMD, the attending health care provider may subsequently prescribe or institute a treatment program. In this manner, the present invention also provides for methods of screening individuals as candidates for treatment of dry AMD. The attending healthcare worker may begin treatment, based on the subject's expression or activity levels of PGC-1α, acetylated PGC-1α, AKT3, PARP2 and/or SIRT-1, before there are perceivable, noticeable or measurable signs of dry AMD in the individual.

The present invention also provides for methods of screening test compounds for their ability to treat dry AMD. The screening methods comprise administering the test compound to abnormal RPE cells and determining expression or activity levels of PGC-1α, AKT3 or SIRT-1 in the abnormal RPE cells in response to the administration of the target compound, wherein an increase in expression or activity levels of PGC-1α, AKT3 or SIRT-1 indicates that the target compound may be effective in treating dry AMD.

The present invention also provides for methods of screening test compounds for their ability to treat dry AMD. The screening methods comprise administering the test compound to abnormal RPE cells and determining expression or activity levels of PARP2 or acetylated PGC-1α in the abnormal RPE cells in response to the administration of the target compound, wherein a decrease in the expression or activity levels of PARP2 or acetylated PGC-1α indicates that the target compound may be effective in treating dry AMD.

The screening methods may also include observation and analysis of phenotypic rescue of the RPE. For example, a compound may be considered an effective target compound if the RPE demonstrate such characteristics, including but not limited to, reduced autophagosomes, reduced lipid droplets, reduced glycogen granules, increased mitochondrial activity and autophagic dynamics, compared to untreated diseased RPE.

As used herein, the term target compound can mean compound which is suspected or not suspected of increasing expression or activity levels of PGC-1α, AKT3 or SIRT-1 in abnormal RPE cells. The term target compound can also mean compound which is suspected or not suspected of decreasing expression or activity levels of acetylated PGC-1α or PARP2 in abnormal RPE cells. The screening methods of the present invention can be performed in an in vitro setting such that the test compound is administered to abnormal RPE cells in culture. Abnormal RPE cells are RPE cells that are not functioning as normal RPE cells, such as but not limited to autophagosome accumulation.

Hallmark morphological changes associated with dry AMD RPE included accumulation of autophagosomes, glycogen granules and lipid droplets as well as disintegrated mitochondria. Functional analysis revealed increased vulnerability to oxidative stress, higher ROS production under oxidative stress, and lower mitochondrial activity in dry AMD RPE compared to normal RPE. Analysis of the autophagic efficiency showed impaired autophagic activity and a lack of response to starvation along with lower autophagic flux in dry AMD RPE.

Specifically, hyperactive mTOR and inactive AMPK signaling in dry AMD RPE contribute to dysregulation of autophagy. In addition, the mechanism that underlies autophagosome accumulation and mitochondrial disintegration in dry AMD RPE is a CRM-1/PGC-1α dependent pathway that is altered due to lower AKT3 expression in dry AMD RPE.

Moreover, CRM-1 overexpression inhibits nuclear localization of PGC-1α and leads to abnormal RPE that display a diseased phenotype resembling those observed in dry AMD RPE. Impaired autophagy caused by mTOR-dependent and independent pathways, e.g., SIRT-1/PGC-1α pathways, in RPE contributes to the pathophysiology of dry AMD.

Despite the high prevalence of dry AMD, to date, there exists no disease altering treatment for dry AMD, and millions of people worldwide continue to suffer from this debilitating disease (Gehrs et al., 2006; Klein et al., 2006; Klein et al., 2011). A major limitation in understanding the pathophysiology of dry AMD is its complexity and the lack of animal models that replicate key features of the human disease (Pennesi et al., 2012). This lack of understanding is primarily due to the multifactorial origin of dry AMD, such as human genetic polymorphisms and countless epigenetic and environmental factors that contribute to the disease. To be clear, dry AMD progression is not equivalent to acute damage to the eye, such as over-exposure to visible or UV light, chemical toxicity and the like.

Consequently, generation of an in vitro disease model that accurately recapitulates the progression of human dry AMD disease phenotypes is of great benefit for exploring and understanding the underlying disease mechanisms. Cultured human RPE from dry AMD donors and controls have characterized herein, and the data show that abnormal RPE that approximate dry AMD RPE exhibit distinct pathological alterations compared to normal RPE. These RPE cells are generated in the absence of genetic manipulation that could erase environmental or epigenetic causes of the disease and can be used as an in vitro model for investigating pathophysiological mechanisms of dry AMD.

By analyzing the RPE isolated from dry AMD and normal donors, specific disease phenotypes characteristic of the disease were identified and this allows for the delineation of distinct functional deficits in dry AMD. In concordance with the EM observations showing autophagosome accumulation, Wong and colleagues have also reported that drusen in dry AMD donor eyes sections contained increased levels of autophagic markers (Wang et al., 2009). Additionally, a recent study reported increased flavoprotein fluorescence, suggesting elevated mitochondrial dysfunction in nonexudative eyes with dry AMD (Field et al., 2012). The functional studies herein elucidate the mechanisms underlying the phenotypes seen by EM, such as accumulation of autophagosomes, lipid droplets, glycogen granules and dysintegrated mitochondria.

The retina is highly susceptible to oxidative stress due to the high levels of oxygen consumption and light-induced oxidative damage. Oxidative stress has long been hypothesized as a key factor in the development of dry AMD (Beatty et al., 2000; Bowes Rickman et al., 2013). The mechanisms underlying the increased susceptibility to oxidative stress in dry AMD, however, remain unclear. The data herein establishes a chronic oxidative stress condition that allows for the study of cell viability of dry AMD RPE and normal RPE up to 48 hrs in the presence of a wide range of H₂O₂ concentrations. The data clearly show that dry AMD RPE exhibit increased susceptibility to chronic oxidative stress. In addition, dry AMD RPE produce increased levels of ROS when exposed to oxidative stress compared to normal RPE.

It is well established that mitochondria are the major source of ROS production and that excess of ROS also induces mitochondrial damage and can lead to diseases (Sena and Chandel, 2012; Rose et al., 2014). Recent studies show that dynamic increase in extracellular ATP accelerates photoreceptor cell apoptosis and that ATP induces photoreceptor death and retinal remodeling in rats (Notomi et al., 2013; Vessey et al., 2014; Yang et al., 2014). The measurement of mitochondrial activity in dry AMD RPE and normal RPE confirmed decreased ATP production by mitochondria, increased ATP production by glycolysis in dry AMD RPE compared to normal RPE, and identified glycolysis as the main source of ATP production in dry AMD RPE and AMD iPSC-RPE. Consistent with these observations, it has recently been shown that in an early phase of hepatocyte failure, an adaptive metabolic shift, from generating energy predominantly from oxidative phosphorylation to glycolysis occurs. This allows maintenance of energy homeostasis during early stages of liver injury, however, leads to hepatocyte dysfunction during terminal stages of chronic liver disease since hepatocytes are unable to sustain high levels of energy production from glycolysis (Nishikawa et al., 2014).

Despite the increasing interest in autophagy and dry AMD within the scientific community, the role of autophagy in dry AMD is poorly understood and the mechanisms by which RPE becomes dysfunctional with aging in dry AMD have not been elucidated. A recent study reported on increased markers of autophagy and exosomes in the drusen of dry AMD donor eyes and speculated that increased autophagy and release of intracellular proteins via exosomes by the aged RPE may contribute to drusen formation and AMD (Wang et al., 2009). Consistent with these morphological observations, the data herein show an increased number of autophagosomes in the dry AMD RPE compared to normal RPE. Mechanistic studies, however, analyzing the ratio of LC3II/LC3I under starvation have demonstrated that despite autophagosme accumulation, the efficiency of autophagy is reduced in dry AMD RPE, and that dry AMD RPE failed to induce autophagy in response to starvation, contrary to what was speculated by Wang et al. LAMP-1 immunostaining clearly demonstrated swollen vesicles indicative of dysfunctional lysosomes in AMD RPE. Together, these observations demonstrate that autophagy is dysfunctional in dry AMD and that the RPE of dry AMD donors are unresponsive to starvation in terms of autophagy induction.

The mTOR pathway plays important roles in age-related diseases (Laplante and Sabatini, 2012; Johnson et al., 2013). A recent publication reported altered mTOR signaling in senescent RPE (Chen et al., 2010). The specific role of mTOR in the pathophysiology of dry AMD, however, has yet to be established.

mTOR is known to inhibit autophagy by phosphorylating ULK-1 and disrupting the interaction between ULK-1 and AMPK (Kim et al., 2011). mTOR and its target protein p70S6K are rapidly and sustainably activated by IGF1 in dry AMD RPE compared to normal RPE. An overactive mTOR pathway can explain the dysfunctional autophagy that was observed in dry AMD RPE.

Analysis of AMPK, an activator of autophagy, revealed that AMPK is inactive in dry AMD RPE as shown by the absence of IGF1 induced AMPK phosphorylation compared to normal RPE. These observations demonstrate that mTOR-dependent mechanisms underlying dysfunctional autophagy in dry AMD RPE could play an important role in the pathogenesis of dry AMD and that its pathway may be targeted for the development of new therapeutics in dry AMD. AKT3 expression and PGC-1α nuclear localization are reduced in dry AMD RPE.

It has been reported that the serine threonine kinase AKT3 controls mitochondrial biogenesis and that silencing the AKT3 gene results in decreased mitochondrial gene expression, mtDNA content, and nuclear-encoded mitochondrial gene transcripts (Wright et al., 2008). In addition, AKT3 silencing results in cytoplasmic accumulation of the master regulator of mitochondrial biogenesis, PGC-1α, and down-regulation of known PGC-1α target genes (Wright et al., 2008). A very recent paper showed that AKT3 controls mitochondrial biogenesis and autophagy via regulation of the major nuclear export protein CRM-1 (Corum et al., 2013).

To understand the mechanisms behind autophagosome accumulation and mitochondrial disintegration, the role of AKT3 gene expression in dry AMD RPE and normal RPE was investigated. The data herein show that AKT3 is down-regulated in dry AMD.

To directly link CRM-1 overexpression and excess of PGC-1α nuclear export to pathophysiological phenotypes observed in dry AMD RPE, human RPE cells were generated from ARPE19 cells by stably overexpressing CRM-1. Morphological analysis was performed and nuclear transport of PGC-1α was verified. The CRM-1 overexpressing cells exhibited disease phenotypes such as accumulation of vacuoles and mitophagosomes, glycogen granules, disintegrated mitochondria, and less dense cytoplasm resembling the disease phenotypes observed in dry AMD RPE. These data therefore suggest that, unlike autophagic dysfunction, which is caused by an overactive mTOR-dependent signaling in dry AMD RPE, autophagosome accumulation is due to down regulation of AKT3 and its downstream target genes, such as PGC-1α. Taken together, these studies identify specific disease phenotypes in human RPE and delineate their underlying mechanisms in dry AMD and demonstrate that dysfunctional autophagy through mTOR dependent and independent (PGC-1α-dependent) pathways contribute to the pathophysiology of dry AMD.

The present invention also provides methods for treating dry AMD in a subject in need thereof, comprising administering at least one compound in an amount effective to increase expression or activity levels of PGC-1α, AKT3 and/or SIRT-1. The present invention also provides methods for treating dry AMD in a subject in need thereof, comprising administering at least one compound in an amount effective to decrease transport of PGC-1α out of the nucleus of a cell.

The present invention also provides methods for treating dry AMD in a subject in need thereof, comprising administering at least one compound in an amount effective to decrease expression or activity levels of PARP2 and/or acetylated PGC-1α.

The present invention also provides methods for treating dry AMD in a subject in need thereof, comprising administering at least one compound in an amount effective to decrease expression or activity of APOB, APOE, APOJ and/or APP.

The present invention also provides methods for treating dry AMD in a subject in need thereof, comprising administering at least one fibrate to the subject. Fibrates are used to modify a subject's lipid profile by activating peroxisome proliferator activated receptor a (PPARα). Examples of fibrates that can be used in the methods of the present invention include but are not limited to bezafibrate, ciprofibrate, clofibrate, gemfibrozil, fenofibrate and clinofibrate.

As used herein, a subject “in need of treatment” is used to mean that the subject is first identified or diagnosed, possibly using the methods of the present invention, as having or becoming susceptible to having dry AMD. In one embodiment, the compound used to increase expression or activity levels of PGC-1α, AKT3 and/or SIRT-1 is a sartan. Sartans, also known as angiotensin receptor blockers or ARBs, are a well-known class of drugs that are known for inhibiting the AT1 receptor for angiotensin II. In specific embodiments, the compound used to increase expression or activity levels of PGC-1α, AKT3 and/or SIRT-1 is losartan, telmisartan, irbesartan, azilsartan, olmesartan, valsartan, eprosartan, temposartan, candesartan, or combinations thereof. A compound known as temposartan is described in PCT Application No. PCT/US2010/049260, which is incorporated by reference. In another embodiment, the compound used to increase expression or activity levels expression or activity levels of PGC-1α, AKT3 and/or SIRT-1 is pyrroloquinoline quinone (PQQ). In another embodiment, the compound used to increase expression or activity levels expression or activity levels of PGC-1α, AKT3 and/or SIRT-1 is telmisartan. In another embodiment, the compound used to decrease transport of PGC-1α out of the nucleus of a cell is leptomycin B, ratjadone, KOS-2464, N-azolylacrylate analogs, FOXO export inhibitors, valtrate, acetoxychavicol acetate, CBS9106, and SINE inhibitors just to name a few.

In still other embodiments, the methods used to increase expression or activity levels of PGC-1α, AKT3 and/or AIRT-1 can include administration of at least one sartan in combination with PQQ. For example, methods used to increase expression or activity levels of PGC-1α, AKT3 and/or SIRT-1 can include administration of at least one of losartan, telmisartan, irbesartan, azilsartan, olmesartan, valsartan, eprosartan, temposartan, candesartan in combination with PQQ. In still other embodiments, the methods used to increase expression or activity levels of PGC-1α, AKT3 and/or SIRT-1 can include administration of at least one sartan in combination with at least one compound that decreases transport of PGC-1α out of the nucleus of a cell. In specific embodiments, the methods may include administration of at least one of losartan, telmisartan, irbesartan, azilsartan, olmesartan, valsartan, eprosartan, temposartan, candesartan in combination with at least one of leptomycin B, ratjadone, KOS-2464, N-azolylacrylate analogs, FOXO export inhibitors, valtrate, acetoxychavicol acetate, CBS9106 or SINE inhibitors. In still other embodiments, the methods used to increase expression or activity levels of PGC-1α, AKT3 and/or SIRT-1 can include administration of PQQ in combination with at least one compound that decreases transport of PGC-1α out of the nucleus of a cell. In specific embodiments, the methods may include administration of PQQ in combination with at least one of leptomycin B, ratjadone, KOS-2464, N-azolylacrylate analogs, FOXO export inhibitors, valtrate, acetoxychavicol acetate, CBS9106 or SINE inhibitors.

In still other embodiments, the methods of treating dry AMD can include methods of inhibiting the expression or activity levels of PARP2 and/or acetylated PGC-1α and/or APOB and/or APOE and/or APOJ and/or APP. PARP family members possess a variety of structural domains, span a wide range of functions and localize to various cellular compartments. Among the molecular actions attributed to PARPs, their role in the DNA damage response (DDR) has been widely documented. In particular, PARPs 1-3 are involved in several cellular processes that respond to DNA lesions, which include DNA damage recognition, signaling and repair as well as local transcriptional blockage, chromatin remodeling and cell death induction. PARP2 expression is increased in dry AMD cell lines, compared to control (normal) cells, and PARP2, in turn, inactivates PGC-1α. For example, methods used to decrease activity or expression of PARP2 include but are not limited to administration of at least one PARP2 inhibitor. PARP2 inhibitors are well known and several are FDA-approved or are being tested and include but are limited to iniparib, talazoparib, olaparib, rucaparib, veliparib and A-966492.

In still other embodiments, the methods of treating dry AMD can comprise increasing expression or activity levels of PGC-1α, AKT3 and/or SIRT-1 and decreasing expression or activity levels of PARP2 and/or acetylated PGC-1α. In specific embodiments, the methods may include co-administration of at least one of losartan, telmisartan, irbesartan, azilsartan, olmesartan, valsartan, eprosartan, temposartan, candesartan in combination with at least one of iniparib, talazoparib, olaparib, rucaparib and veliparib. In still other embodiments, the methods of treating dry AMD may also comprise increasing expression or activity levels of PGC-1α, AKT3, AMPK and/or SIRT-1 and decreasing transport of PGC-1α out of the nucleus of a cell, as well as decreasing expression or activity levels of PARP2 and/or acetylated PGC-1α. In specific embodiments, the methods of treatment of dry AMD may include co-administration of one or more of of at least one of losartan, telmisartan, irbesartan, azilsartan, olmesartan, valsartan, eprosartan, temposartan, candesartan in combination with at least one of iniparib, talazoparib, olaparib, rucaparib and veliparib, in combination with leptomycin B, ratjadone, KOS-2464, N-azolylacrylate analogs, FOXO export inhibitors, valtrate, acetoxychavicol acetate, CBS9106 or SINE inhibitors, in combination with at least one of fenofibrate, ciprofibrate, bezafibrate, clofibrate, gemfibrozil and clinofibrate.

In other specific embodiments, the methods of treatment of dry AMD may include co-administration of one or more of of at least one of a fibrate with at least one compound that increases expression or activity levels of PGC-1α, AKT3, AMPK and/or SIRT-1, or in combination with at least one compound that decreases expression or activity of PARP2 and/or acetylated PGC-1α.

The dose of telmisartan, alone or in combination, for oral administration can be from about 0.1 to about 5 mg/kg body weight/day. The dose of telmisartan, alone or in combination, for ocular injection or implant, e.g., in the subretinal zone can be from about 30 to about 50 μg. The dose of telmisartan, alone or in combination, for topical administration, e.g., an eye drop, can be from about 10 to about 50 μg/day.

The dose of temposartan, alone or in combination, for oral administration can be from about 0.1 to about 5 mg/kg body weight/day. The dose of temposartan, alone or in combination, for ocular injection or implant, e.g., in the subretinal zone can be from about 30 to about 50 μg. The dose of temposartan, alone or in combination, for topical administration, e.g., an eye drop, can be from about 10 to about 50 μg/day.

The dose of candesartan, alone or in combination, for oral administration can be from about to about 32 mg/day. The dose of candesartan, alone or in combination, for ocular injection or implant, e.g., in the subretinal zone, can be from about 0.1 to about 4 μg. The dose of candesartan, alone or in combination, for topical administration, e.g., an eye drop can be from about 0.1 to about 3.2 μg/day.

The dose of PQQ, alone or in combination, for oral administration can be from about 100 to about 2000 mg/day. The dose of PQQ, alone or in combination, for ocular injection or implant, e.g., in the subretinal zone, can be from about 0.06 to about 0.2 mg. The dose of PQQ, alone or in combination, for topical administration, e.g., an eye drop can be from about 0.01 to about 0.5 mg/day.

The dose of fenofibrate, alone or in combination, for oral administration can be from about 0.1 to about 100 mg/kg body weight/day. The dose of fenofibrate, alone or in combination, for ocular injection or implant, e.g., in the subretinal zone can be from about 1 to about 50 ng. The dose of fenofibrate, alone or in combination, for topical administration, e.g., an eye drop, can be from about 1 to about 50 ng/day.

The dose of bezafibrate, alone or in combination, for oral administration can be from about 0.1 to about 100 mg/kg body weight/day. The dose of bezafibrate, alone or in combination, for ocular injection or implant, e.g., in the subretinal zone can be from about 1 to about 50 ng. The dose of bezafibrate, alone or in combination, for topical administration, e.g., an eye drop, can be from about 1 to about 50 ng/day.

The dose of ciprofibrate, alone or in combination, for oral administration can be from about 10 to about 100 mg/day. The dose of ciprofibrate, alone or in combination, for ocular injection or implant, e.g., in the subretinal zone can be from about 1 to about 50 ng. The dose of ciprofibrate, alone or in combination, for topical administration, e.g., an eye drop, can be from about 1 to about 50 ng/day.

The dose of olaparib, alone or in combination, for oral administration can be from about 10 to about 100 mg/day. The dose of olaparib, alone or in combination, for ocular injection or implant, e.g., in the subretinal zone can be from about 1 to about 100 ng. The dose of olaparib, alone or in combination, for topical administration, e.g., an eye drop, can be from about 1 to about 50 ng/day.

The dose of rucaparib, alone or in combination, for oral administration can be from about 100 to about 200 mg/day. The dose of rucaparib, alone or in combination, for ocular injection or implant, e.g., in the subretinal zone can be from about 1 to about 100 ng. The dose of rucaparib, alone or in combination, for topical administration, e.g., an eye drop, can be from about 1 to about 50 ng/day.

The dose of veliparib, alone or in combination, for oral administration can be from about 100 to about 200 mg/day. The dose of veliparib alone or in combination, for ocular injection or implant, e.g., in the subretinal zone can be from about 1 to about 100 ng. The dose of veliparib, alone or in combination, for topical administration, e.g., an eye drop, can be from about 1 to about 50 ng/day.

The dose of UPF 1069, alone or in combination, for oral administration can be from about 10 to about 200 mg/day. The dose of UPF 1069 alone or in combination, for ocular injection or implant, e.g., in the subretinal zone can be from about 1 to about 100 ng. The dose of UPF 1069, alone or in combination, for topical administration, e.g., an eye drop, can be from about 1 to about 50 ng/day.

The dose of A-966492, alone or in combination, for oral administration can be from about 10 to about 200 mg/day. The dose of A-966492 alone or in combination, for ocular injection or implant, e.g., in the subretinal zone can be from about 1 to about 100 ng. The dose of A-966492, alone or in combination, for topical administration, e.g., an eye drop, can be from about 1 to about 50 ng/day.

The dose of talazoparib, alone or in combination, for oral administration can be from about 10 to about 200 mg/day. The dose of talazoparib alone or in combination, for ocular injection or implant, e.g., in the subretinal zone can be from about 1 to about 100 ng. The dose of talazoparib, alone or in combination, for topical administration, e.g., an eye drop, can be from about 1 to about 50 ng/day.

The efficacy of treatment can be monitored by typical ophthalmological procedures, such as but not limited to monitoring the size and/or number of drusen, as well as testing visual acuity.

A pharmaceutical composition of the invention is formulated to be compatible with its intended route of administration. Examples of routes of administration include oral and parenteral (e.g., intravenous, intradermal, subcutaneous, inhalation, topical (transdermal or eye drops), transmucosal and rectal administration). Solutions or suspensions used for parenteral, topical intradermal or subcutaneous application can include, but are not limited to, a sterile diluent such as water for injection, saline solution, fixed oils, polyethylene glycols, glycerine, propylene glycol or other synthetic solvents, antibacterial agents such as benzyl alcohol or methyl parabens, antioxidants such as ascorbic acid or sodium bisulfite, chelating agents such as ethylenediaminetetraacetic acid, buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable or topical use include sterile aqueous solutions (where water soluble) or dispersions and sterile powders for the extemporaneous preparation of sterile injectable or topical solutions or dispersion. For intravenous administration, suitable pharmaceutical carriers include physiological saline, bacteriostatic water, Cremophor EL™ (BASF) or phosphate buffered saline (PBS). In all cases, the compositions must be sterile and should be fluid to the extent that easy syringeability or eye drop formation exists. It must be stable under the conditions of manufacture and storage and must be preserved against the contaminating action of microorganisms such as bacteria and fungi. The pharmaceutical carrier can be a solvent or dispersion medium containing, for example, water, ethanol, polyol (for example, glycerol, propylene glycol and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it may be desirable to include isotonic agents, for example, sugars, polyalcohols such as manitol, sorbitol, sodium chloride in the composition. Prolonged absorption of the injectable or topical compositions can be brought about by including in the composition an agent which delays absorption, for example, aluminum monostearate and gelatin.

Sterile solutions can be prepared by incorporating the active compound/composition in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutical carrier. They can be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition. The tablets, pills, capsules, troches and the like may contain any of the following ingredients, or compounds of a similar nature, such as but not limited to a binder, such as microcrystalline cellulose, gum tragacanth or gelatin, an excipient such as starch or lactose, a disintegrating agent such as alginic acid, Primogel or corn starch, a lubricant such as magnesium stearate or Sterotes, a glidant such as colloidal silicon dioxide, a sweetening agent such as sucrose or saccharin, or a flavoring agent such as peppermint, methyl salicylate or flavoring.

In one embodiment, the active compound is prepared with pharmaceutical carriers that will protect the composition against rapid elimination from the body, such as a controlled release formulation. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These compositions can be prepared according to methods known to those skilled in the art, for example, as described in U.S. Pat. No. 4,522,811. It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active complex calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The pharmaceutical compositions can be included in a container, pack or dispenser together with instructions for administration.

The examples provided herein are illustrative of select embodiments of the invention and are not to intended to limit the scope of the invention.

### Example 1

Materials and Methods

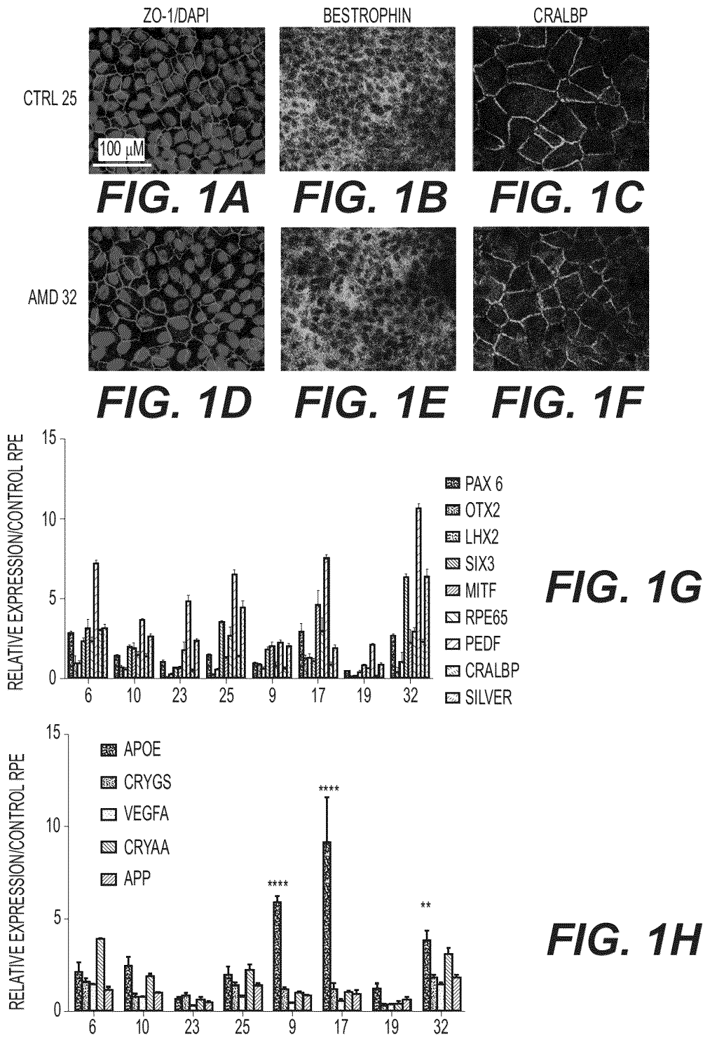

FIGS. 1-3 show various representations of an in vitro disease model of AMD. A total of 10 eyes from 5 organ donors clinically diagnosed with dry AMD and 10 eyes from 5 clinically normal donors were purchased from Lions Medical Eye Bank. RPE were isolated according to established protocols (Maminishkis et al., 2006) and cultured in Epithelial Cell Media (EpiCM, ScienCell) under controlled oxygen (5%) and CO2 (5%) conditions. Studies have shown that low (physiological) oxygen concentration promotes RPE growth (Knorr et al., 1993) and better protects from ROS-induced damage in vitro. RPE were then purified with Magnetic-Activated Cell Sorting (MACS) by positive selection for epithelial cells using anti-BEST1 antibody (Abcam) and anti-E-cadherin (Miltenyi Biotech); and by negative selection using a fibroblast-specific antibody (Miltenyi Biotech) to remove fibroblasts. The purity of the sorted cells was confirmed by immunostaining with anti-ZO-1 and anti-BEST1, and Real-Time PCR for RPE specific genes. The RPE cells were grown in Epithelial Cell Media (EpiCM, ScienCell) at 37° C., with 5% O₂ and 5% CO₂. FIG. 2 shows that the RPE cells were dedifferentiated to iPSC, followed by redifferentiation to RPE-iPSC-RPE. Skin fibroblasts from a dry AMD patient were used to generate iPSC-RPE. Genotyping of the donors and the patient is represented in FIG. 3.

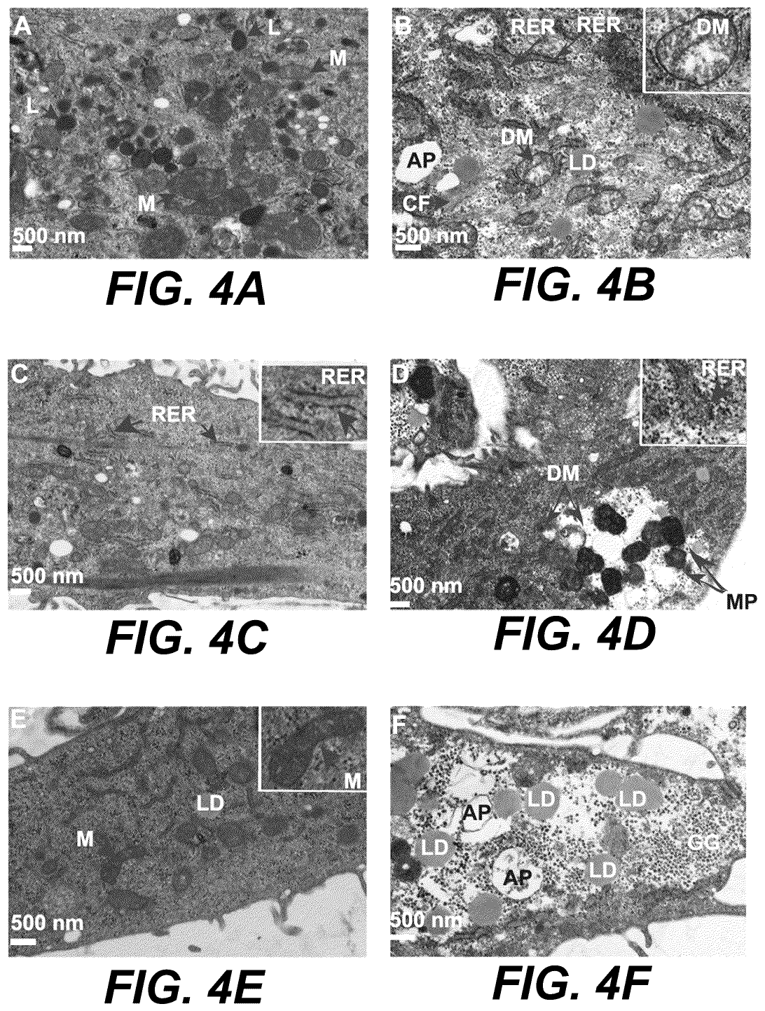

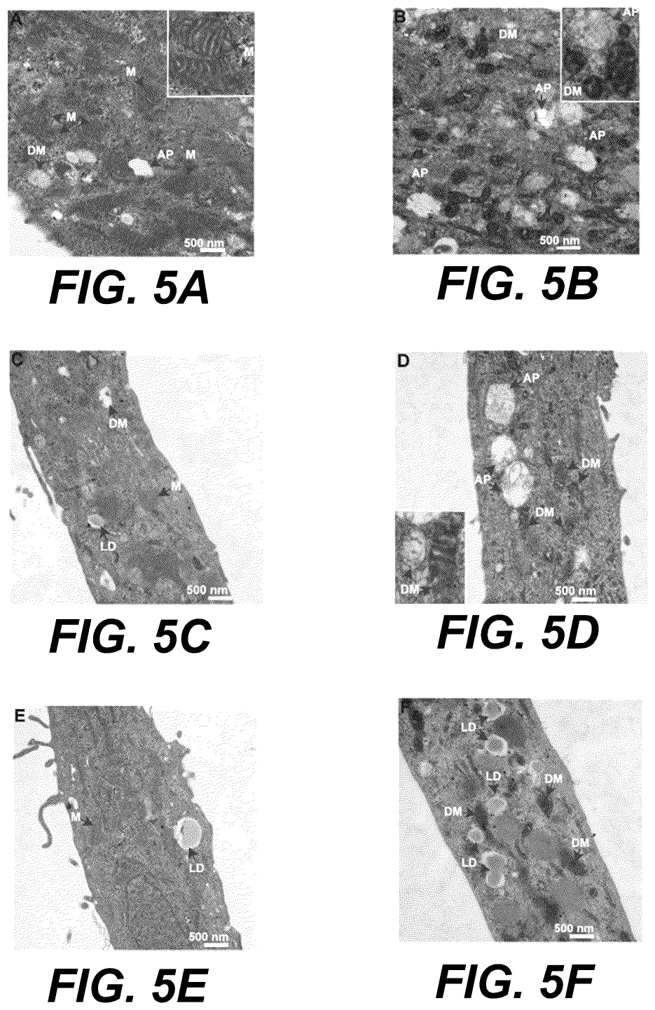

Electron Microscopy of RPE and iPSC RPE are shown in FIGS. 4 and 5, respectively. RPE cell cultures were rinsed with Dulbecco PBS (without Ca²⁺ and Mg²⁺), fixed twice in PBS-buffered glutaraldehyde (2.5% at pH 7.4) and PBS-buffered osmium tetroxide (0.5%), and embedded in epoxy resin. Thin sections (90 nm) were collected on 200 μm mesh copper grids, dried for 24 hours, and double-stained with uranyl acetate and lead citrate. Sections were viewed and photographed with JEOLJM-1010 electron microscope (Kokkinaki et al., 2013).

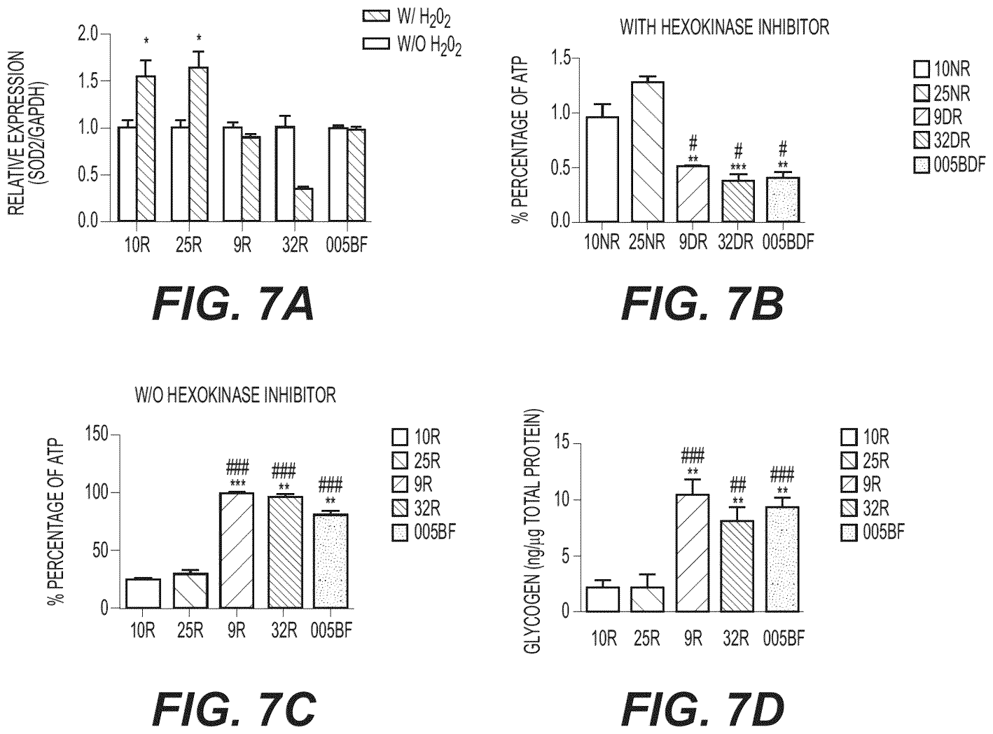

The results from a glycogen concentration assay are shown in FIG. 7D. Cytoplasmic accumulation of glycogen was assayed using the Glycogen Assay Kit (Sigma) on RPE monolayers grown in 96-well plates. Glycogen concentration was determined by a coupled enzyme assay, which produces a colorimetric (570 nm)/fluorometric (535/587 nm) product, proportional to the glycogen present.

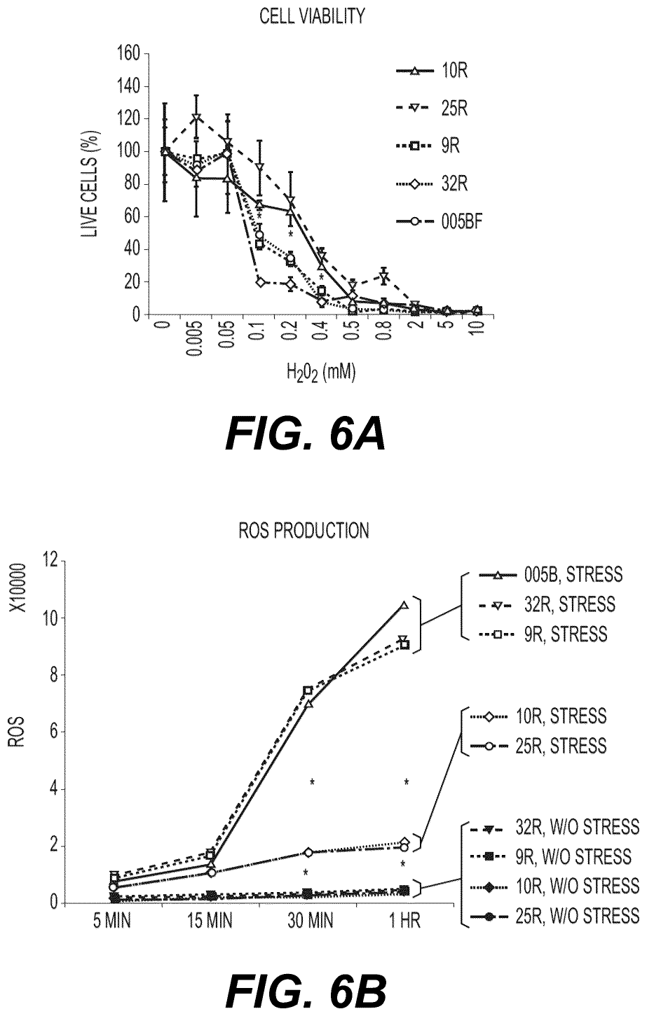

The results from an oxidative stress assay are shown in FIG. 6A, 6B and 7A. RPE were cultured at 80-90% confluency in 96-well plates, at 37° C. with 5% O₂ and 5% CO₂. Oxidative stress was induced with different concentrations of H₂O₂ ranging from 0-10 mM for 24 or 48 hrs, followed by cell viability measurements.

Cell viability assay results are shown in FIG. 6A. Cell viability was measured following a 30 min incubation with the PestoBlue Reagent (Life Technologies) of RPE monolayers cultured on 96-well plates, 6 wells used for each sample. Fluorescence measurements were performed using an Ultra384 plate reader with 535 nm excitation and 612 nm emission wavelengths.

ROS measurements are shown in FIG. 6B. Production of Reactive Oxygen Species (ROS) under oxidative stress was measured using the OxiSelect™ Intracellular ROS Assay Kit (Cell Biolabs). RPE monolayers were cultured in 96-well plates in serum free EpiCM for 20 h and then loaded with 1 mM of the cell-permeable fluorogenic probe 2′-7′-Dichlorodihydrofluorescin diacetate (DCFH-DA) for 1 h. Oxidative stress was induced with 2 h incubation in 0.4 mM H₂O₂ . The fluorescence intensity of each sample, proportional to the ROS levels, was measured against the fluorescence of the provided standard at the indicated time-points using a Tecan (Morrisville, NC) Ultra 384 plate reader.

ATP levels are shown in FIG. 7B, 7C. ATP levels were measured with the Mitochondrial ToxGlo Assay (Promega). To assay the mitochondrial activity in dry AMD and normal RPE, the ATP measurement was performed following 2 h incubation with 10 μM of the bromopyruvate analogue (3-BrPA), an inhibitor of the glycolytic enzyme hexokinase II (EMD Millipore).

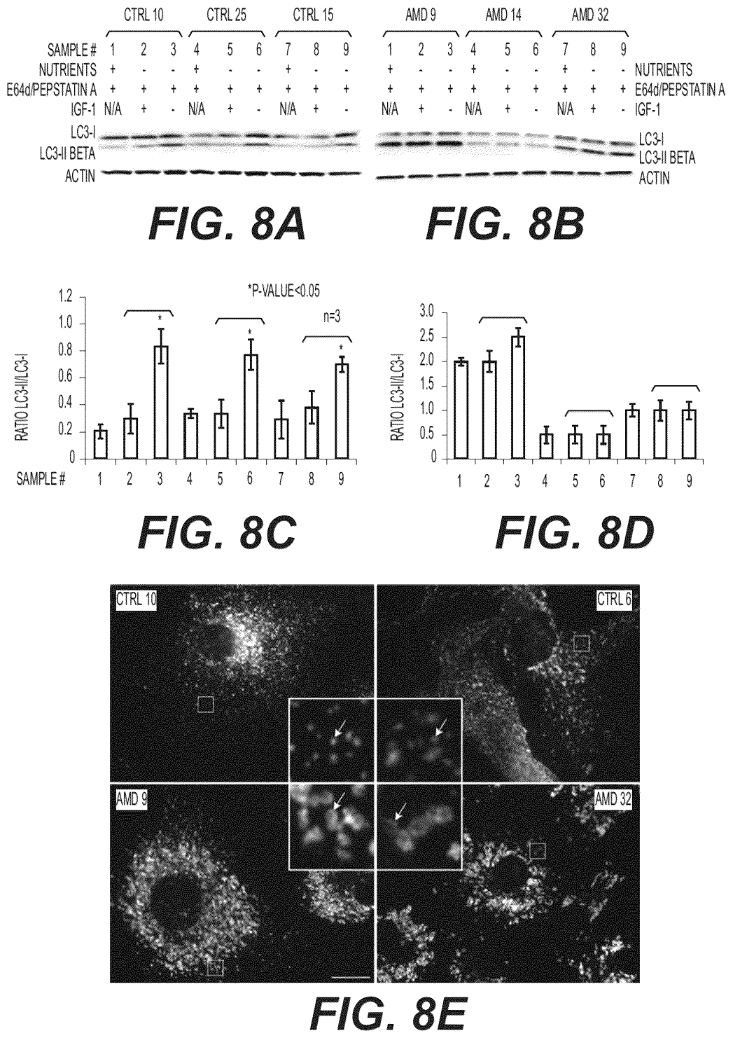

The results from an autophagy dynamics assay are shown in FIG. 8A-8D. For measurement of LC3-II/LC3-I ratios, normal and dry AMD RPE cells were pre-treated with IGF1 for 1 h and starved in HBSS for 4 h in the presence of IGF1; lysosomal inhibitors E64d and Pepstatin A were added in the last 2 h of starvation at a concentration of 10 μg/ml and the cells were lysed and analyzed with immunoblot for LC3 and p62 based on the established protocol (Tanida et al., 2005).

Starvation and treatment with IGF1. In all experiments including starvation, the RPE were incubated for 20 h in serum-free EpiCM media (ScienCell) and subsequently starved in HBSS for 4 h. IGF1 (R&D Systems) was added at 75 ng/ml during the indicated time intervals for each experiment.

Antibodies. a) Primary antibodies. The primary antibodies used were as follows: rabbit anti-mTOR, rabbit anti-phospho-mTOR(Ser2448), rabbit anti-p70S6K, rabbit anti-phospho-p70S6K(Thr389), rabbit anti-G L, rabbit anti-AMPK, and rabbit anti-phospho-AMPKα from Cell Signaling Technology; rabbit anti-CRM-1 from Abcam; rabbit anti-H3 histone from Cell Signaling Technology; rabbit anti-PGC-1α from Santa Cruz Biotech.; rabbit anti-LC3 from Thermo Fisher Scientific; rabbit anti-p62/SQSTM1 and rabbit anti-beta actin from Cell Signaling Technology; mouse anti-LAMP-1 from BD Biosciences; mouse anti-BEST1 from Abcam; mouse anti-ZO-1 from Invitrogen; fibroblast-specific antibody (conjugated to magnetic beads), mouse anti-E-cadherin (CD324) from Miltenyi Biotech and mouse anti-Flag from Origene Technologies.

b) Secondary antibodies. The secondary antibodies used were as follows: goat anti-rabbit HRP-linked from Cell Signaling Technology; donkey anti-mouse Alexa488-linked and donkey anti-mouse Alexa594-linked from Invitrogen; and anti-Mouse IgG MicroBeads from Miltenyi Biotech.

Immunoblot analysis. Protein samples were extracted in radioimmunoprecipitation assay (RIPA) buffer (1% NP-40, 0.5% sodium deoxycholate, and 1% SDS in 1×PBS), containing freshly added Protease and Phosphatase Inhibitor Cocktail Tablets (Roche Applied Science), 1×Protease Inhibitor Cocktail Set I (EMD Millipore), 1 mM sodium vanadate, 50 mM sodium fluoride, and 1 mM PMSF (Sigma-Aldrich). Protein concentrations were measured by Bradford assay (Bio-Rad). Protein samples were analyzed using the NuPAGE electrophoresis and XCell Western blot system (Invitrogen). Primary and secondary antibodies were used based on the manufacturer's instructions. Immunoreactive protein bands were visualized by the Clarity chemiluminescent substrate (Biorad) followed by imaging with the MyECL imager (Thermo Fisher Scientific). Densitometry was performed using the ImageJ software.

Immunostaining. RPE cells grown on plastic eight-well chamber slides (Thermo Fisher Scientific) were stained using established protocols for the primary and secondary antibodies. Stained cells were mounted with anti-fading medium (Invitrogen), and imaged using an EVOS FL microscope (Life Technologies).

Quantitative Real-Time Polymerase Chain Reaction Analyses. Total RNA was extracted with the RNeasy kit (Qiagen), treated with RNase-free DNase I (Qiagen), and reverse transcribed with oligo-dT using the SuperScript III cDNA synthesis kit (Invitrogen). Quantitative PCR was performed with the QuantiTect SYBR Green PCR Kit (Qiagen). Specific primers for each gene were designed with the PrimerQuest software (Integrated DNA Technologies), and the cDNA sequences of each gene (GenBank) were used to produce 100-250 bp PCR amplicons that span one or more exon/intron boundaries.

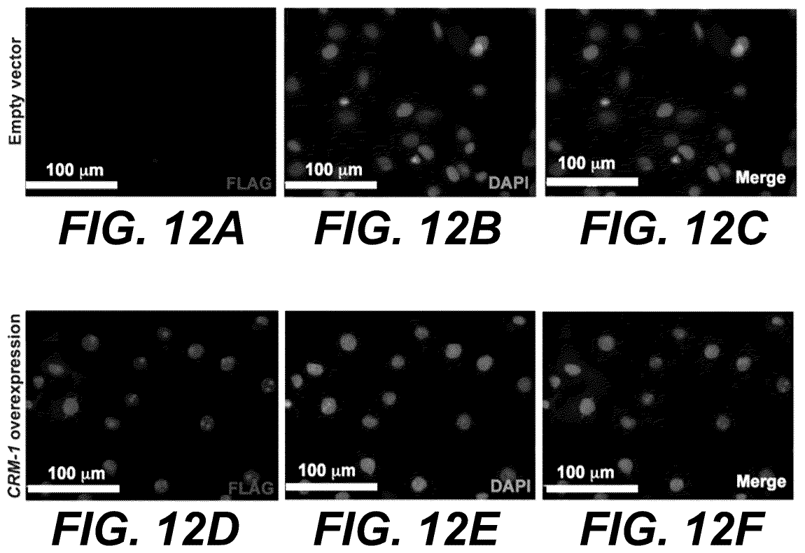

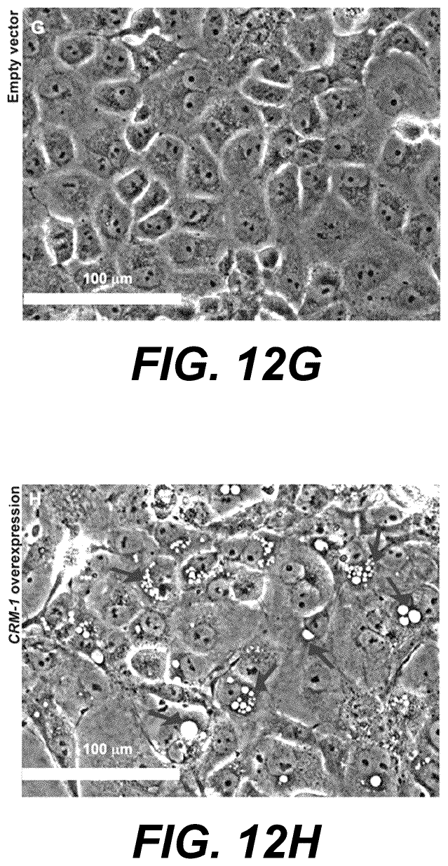

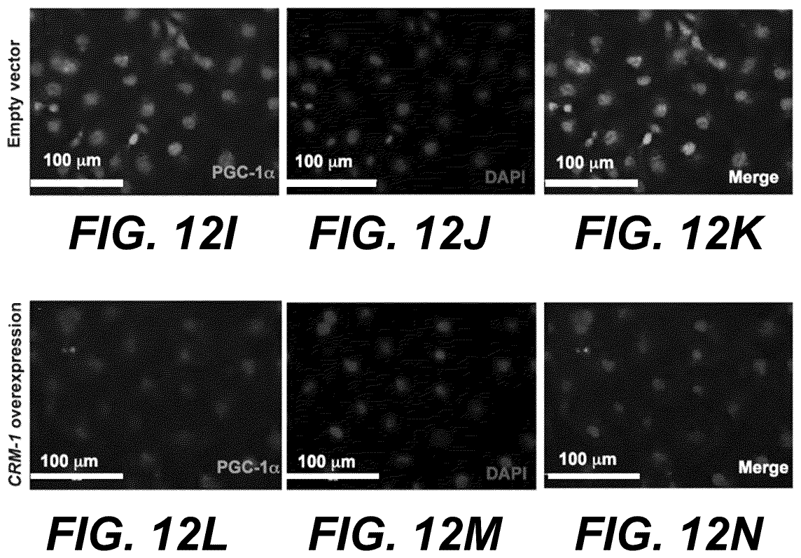

Generation of CRM-1 overexpressing RPE is seen in FIG. 12. ARPE19 cells were transfected by nucleofection with the pCMV6-Entry vector (Origene Technologies) or with the CRM-1 overexpressing plasmid (Flag-CRM1 in pCMV6-Entry from Origene Technologies). 5×10⁵ cells and 1 μg of plasmid DNA were used in each nucleofection performed with the Amaxa Biosystems Nucleofector II (Lonza, Allendale, NJ) using the high efficiency I-013 protocol and the Basic Nucleofector Kit for Primary Mammalian Epithelial Cells Solution Mix (Cat. No VPI-1005) from Lonza. 48 hrs after the transfection G-418 (Sigma) was used at a concentration of 1.5 mg/ml to select the transfected cells. Approximately 90% of the cells died in the first two weeks of selection and the cells that survived formed resistant colonies in the presence of G-418. After 3 weeks of selection, the transfected cells were analyzed by immunostaining with anti-PGC-1α, inverted microscope and electron microscopy for morphological analysis.

Statistical analysis. Quantitative functional and gene expression assays were performed 3 times, each sample represented by 3-6 replicas per experiment. Mean averages±standard deviations were calculated and the statistical significance of the observed differences was tested by t-test, using p-value<0.05.

Results

The study of dry AMD is challenging due to the lack of an in vitro model that accurately recapitulates key features of the human disease. Animal models are of limited value as they fail to replicate human genetic polymorphisms and long-term exposure to environmental factors (Lim et al., 2012) that induce epigenetic changes.

To overcome this limitation, an in vitro human disease model was developed by isolating RPE from 5 dry AMD donors (AMD RPE) and compared their phenotypes and functions to the RPE isolated from 5 normal donors (normal RPE) and generation of respective iPSC-RPE. To establish cultures, human RPE cells were purified by magnetic cell sorting (MACS), using specific antibodies for positive and negative selection of epithelial cells as described in the Materials and Methods. The purified human RPE cells were cultured under 5% oxygen concentration to avoid oxidative stress and analyzed for expression of specific RPE proteins and genes as shown in FIG. 1A-B. In addition, single nucleotide polymorphism (SNP) analysis was performed to identify mutations in protective and risk alleles in RPE genomic DNA from donors (FIG. 2). Interestingly, dry AMD donor #9, which had a protective allele, nevertheless developed AMD, possibly due to heavy smoking (long-term smoking of more than 20 cigarettes per day), providing further evidence for the multifactorial origin of dry AMD. The iPSC were generated from RPE of AMD and normal donors and from skin fibroblasts of a dry AMD patient according to the established protocol using non-integrating sendai viruses. The iPSC were able differentiate to RPE using the established protocol as previously described (Kokkinaki. M., et al., Stem Cells, 29(5):825-835 (2011)).

Electron microscopy (EM) imaging (FIGS. 4, 5) revealed that all 5 dry AMD RPE cultures and the corresponding iPSC-RPE exhibited distinct disease phenotypes compared to the normal RPE. The dry AMD RPE contained less dense cytoplasm, a higher content of lipid droplets, increased number of glycogen granules, and enlarged autophagosomes. The space between the two membranes of the rough endoplasmic reticulum (RER) appeared to be wider and irregular in dry AMD RPE (FIGS. 3B and 3F), whereas it appeared rather uniform in normal RPE. Cytoskeletal filaments formed fascicles and appeared condensed and mitochondria appeared disintegrated in dry AMD RPE compared to normal RPE. Quantification of lipid droplets and glycogen granules by fluorescent staining and colorimetric assay respectively revealed higher levels of lipid droplets and glycogen granules in dry AMD RPE compared to normal RPE, further confirming the EM phenotypes. The distinct phenotypes identified in dry AMD RPE support the use of these cells as an in vitro model to study the underlying mechanisms responsible for the observed phenotypes.

RPE cells are constantly exposed to light-induced oxidative stress (Cai et al., 2000). Over time, this exposure may damage RPE tight junctions and disrupt the retinal blood barrier (Negi and Marmor, 1984). To assess the functional consequences of the observed phenotypes, chronic oxidative stress conditions were established that would allow long-term RPE cells culture using a series of increasing H₂O₂ concentrations. Under these conditions, RPE cells can be cultured up to 48 hrs with increasing doses of H₂O₂ to study their susceptibility to oxidative stress.

Cell viability assays using 0.2-10 mM of H₂O₂ for 24 and 48 hrs revealed that dry AMD RPE, AMD iPSC-RPE and normal RPE are affected differently by oxidative stress. The data demonstrated that while H₂O₂ treatment for 24 hrs only partially separated the dry AMD RPE and AMD iPSC-RPE from normal RPE (FIG. 6A), 48 hrs of H₂O₂ treatment revealed significantly higher vulnerability for AMD RPE (p-value: 0.01). The dry AMD RPE died faster and at a lower concentration of H₂O₂ compared to normal RPE (FIG. 6A).

To examine whether structural defects observed in mitochondria (FIG. 6B) correlate with elevated oxidative stress, ROS production was measured in the presence of 0.4 mM H₂O₂ for 5 min to 1 hr in dry AMD RPE and normal RPE. The data showed that dry AMD RPE produce significantly higher ROS levels than normal RPE under oxidative stress (FIG. 6B).

ATP levels were measured to analyze mitochondrial activity in dry AMD and normal RPE. This assay revealed that total ATP levels were higher in AMD RPE compared to normal RPE (FIG. 7C). When the cells were with hexokinase inhibitor to block the glycolytic ATP production, the ATP produced by mitochondria was significantly lower in dry AMD RPE (FIG. 7B). Conversely, ATP levels measured in the absence of hexokinase inhibitor revealed that glycolytic ATP production is significantly higher in dry AMD RPE compared to normal RPE (FIG. 7C) showing that, in AMD RPE, ATP is produced primarily through glycolysis.

To investigate the biological relevance of autophagosome accumulation observed by the EM data and to directly study the role of autophagy in the pathophysiology of dry AMD, autophagy was measured by inducing nutrient starvation in the presence of lysosomal inhibitors E64d/Pepstatin A and in the presence or absence of the insulin growth factor (IGF1). A commonly accepted method to monitor autophagic flux is to assay ubiquitin-like microtubule-associated protein 1 light chain LC3-I, which after lipidation becomes LC3-II, is inserted into the inner and outer membranes of the autophagosome and is finally degraded in the lysosomes (Kimmelman, 2011) (Mizushima, 2004), (Mizushima and Yoshimori, 2007; Troncoso et al., 2012). To obtain an accurate measurement of the endogenous levels of LC3-II, the dry AMD and normal RPE were pre-treated with lysosomal inhibitors E64d and pepstatin A (Tanida et al., 2005) to inhibit lysosomal degradation. Under these conditions the levels of the lipidated form of LC3-II are an accurate measurement of autophagic flux. The results revealed that normal RPE rapidly induced autophagy after starvation only in the absence of IGF1, whereas the dry AMD RPE had higher levels of LC3-II, independently of IGF1 treatment, and starvation, with or without IGF1, did not increase the ratio of LC3-II/LC3-I. Therefore, the addition of IGF1, expected to suppress autophagy through activation of the AKT/mTOR signaling, did not decrease the ratio of LC3-II/LC3-I in AMD RPE (FIG. 8A, 8B), as shown by the western blots of three independent experiments (FIGS. 8C, 8D). These observations suggest that pathways that regulate autophagy are impaired in dry AMD RPE.

Since lysosomal activity plays an important role in regulating autophagic flux, lysosomal enzyme activity was measured in dry AMD RPE and normal RPE. Late autophagic vesicles, autolysosomes, were analyzed by immunostaining with lysosomal-associated membrane protein 1 (LAMP-1) antibody in the dry AMD RPE and normal RPE cultures. The data showed that LAMP-1-positive organelles in ry AMD RPE are enlarged and annular, as opposed to the smaller discrete puncta observed in normal RPE (compare arrowed structures in magnified insets in FIG. 8E). This enlarged morphology suggests inefficient degradation of cellular debris within the lysosomes of dry AMD RPE. These observations strongly suggest dysfunctional autophagy in dry AMD RPE that translates into lower autophagic flux and debris accumulation in the cytoplasm.

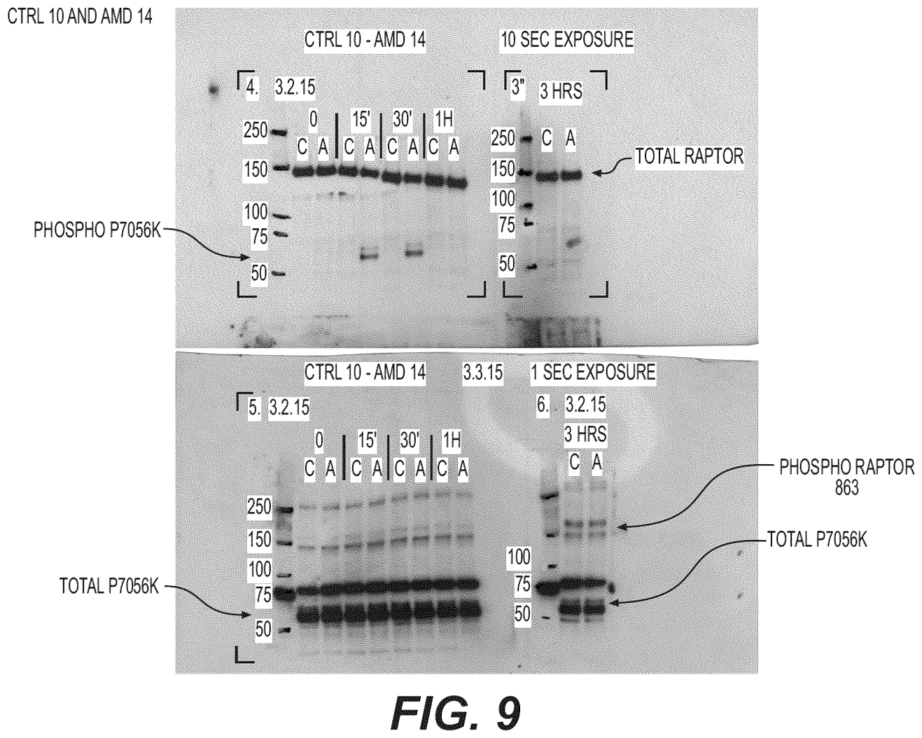

AMD RPE and four normal RPE cultures were starved and treated with IGF1, and mTOR activity was measured by analyzing the levels of mTOR phosphorylation at Ser2448 and the mTOR target ribosomal protein S6 kinase (p70S6K) phosphorylation at Thr389. The elevated mTOR activity in dry AMD RPE was confirmed by analyzing the levels of phospho-p70S6K (Thr389). AMD RPE cultures tested (FIG. 9) showed a rapid activation of p70S6K after 15 min of IGF1 addition that was sustained until 1 hr, and decreased after 1 hr to reach a low level after 3 h. The normal RPE (n=4), however, showed significantly lower p70S6K activation or no activation after the addition of IGF1 that remained the same until 3 h, similar to the deactivation levels observed in dry AMD RPE.

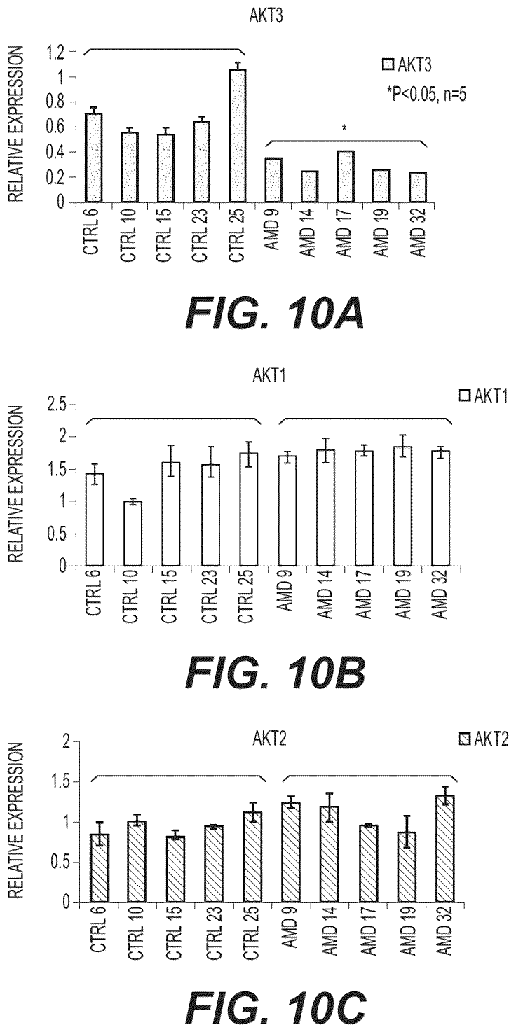

To determine if the autophagosome accumulation observed in dry AMD RPE could be related to lower AKT3 gene expression, mRNA levels were compared in normal and dry AMD RPE by real-time qPCR. Interestingly, all dry AMD RPE showed significantly lower AKT3 gene expression levels compared to normal RPE, whereas AKT1 and AKT2 levels were not significantly different in dry AMD RPE compared to normal RPE (FIG. 10A).

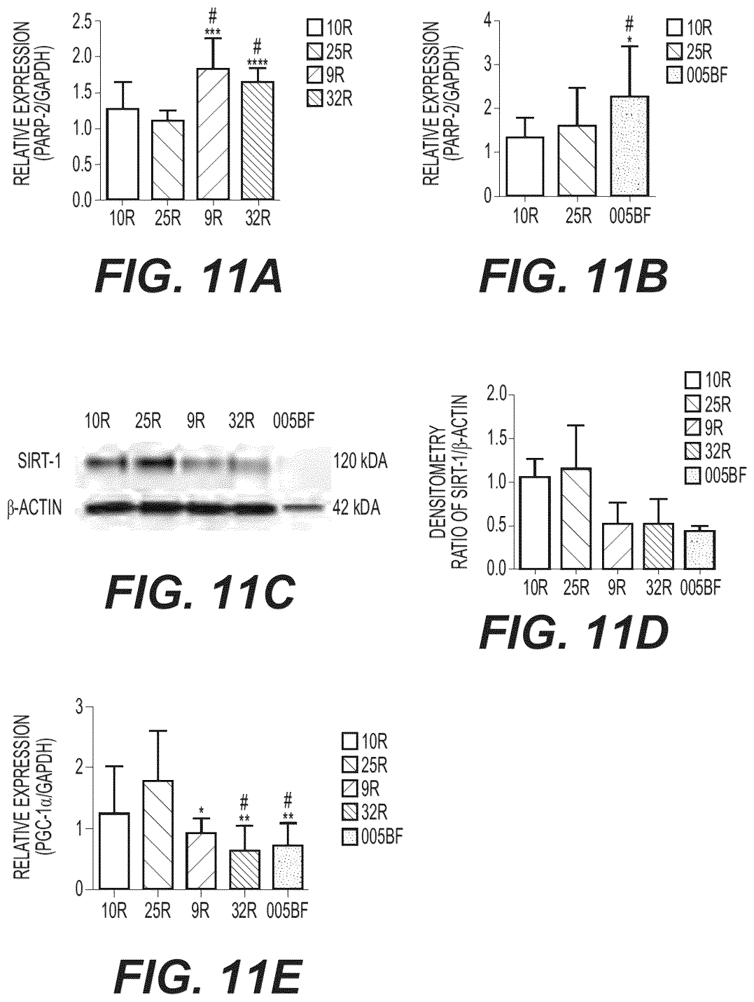

Based on the observations on mitochondrial disintegration (FIGS. 4B-4F, 5B-5F) and dysfunction in AMD RPE, AMD RPE-iPSC-RPE and AMD Skin-iPSC-RPE, the implication of the pathways regulators of mitochondrial biogenesis and functions was investigated. RNA sequencing was performed on 5 native AMD RPE and 5 native normal RPE cultured from AMD and normal donors (FIG. 3). RNA sequencing revealed that PARP2 expression levels are significantly increased (1.35 fold increase, p=0.028) in AMD RPE as compared to normal RPE. To further confirm the RNA sequencing data, quantitative Real Time PCR was performed on AMD RPE-iPSC-RPE and normal RPE-iPSC-RPE generated from the donors native RPE. The Real Time PCR data supported the RNA sequencing results and showed a significant increase in the levels of PARP2 expression in AMD RPE-iPSC-RPE as compared to normal RPE-iPSC-RPE (FIG. 11A). PARP2 expression was also verified in AMD Skin-iPSC-RPE as compared to normal RPE-iPSC-RPE. Interestingly, PARP2 expression levels were also increased in dry AMD Skin-iPSC-RPE as compared to normal RPE-iPSC-RPE (FIG. 11B, asterisk (*) and hashtag (#) represent p≤0.05 of mean±S.E. of three independent experiments).

SIRT-1 protein expression was examined by western blot analysis in all generated cells lines. A decrease in SIRT-1 protein levels was observed in AMD RPE-iPSC-RPE and AMD-Skin-iPSC-RPE as compared to normal RPE-iPSC-RPE (FIG. 11C). Densitometry analysis of three independent western blot experiments showed about a 2-fold decrease in SIRT-1 protein in AMD RPE-iPSC-RPE and AMD Skin-iPSC-RPE as compared to normal RPE-iPSC-RPE (FIG. 11D).

PGC-1α expression was analyzed by Real Time PCR. PGC-1α expression levels were also decreased in AMD RPE-iPSC-RPE and AMD-Skin-iPSC-RPE as compared to normal RPE-iPSC-RPE (FIG. 11E, (asterisk (*) and hashtag (#) represent p≤0.05 of mean±S.E. of three independent experiments).

To show that the pathological phenotypes observed in dry AMD RPE are linked to increased CRM-1 expression causing PGC-1α nuclear export, human RPE cells (ARPE19) were transfected with a CRM-1 overexpressing plasmid (Flag-CRM-1 in pCMV6-Entry) to generate stable cell lines. As a control, ARPE19 cell lines transfected with empty vector pCMV6-Entry were also generated. FIG. 12D shows Flag-CRM-1 overexpression detected by anti-Flag immunostaining. To verify the effect of CRM-1 overexpression on PGC-1α nuclear localization, ARPE19 cells overexpressing CRM-1 were immunostained with anti-PGC-1α three weeks after transfection. As shown in FIG. 12L, PGC-1α nuclear localization is significantly reduced compared to control cells (FIG. 12I). The CRM-1 overexpressing ARPE19 cells were then analyzed with inverted microscope for any morphological changes compared to control cells. FIG. 12H shows a representative phase contrast image of the ARPE19 cells overexpressing CRM-1, compared to ARPE19 transfected with empty control vector (FIG. 12G). CRM-1 overexpressing cells show accumulation of multiple vacuoles of various sizes whereas ARPE19 control cells appear normal and devoid of these vacuoles (FIGS. 12G, 12H).

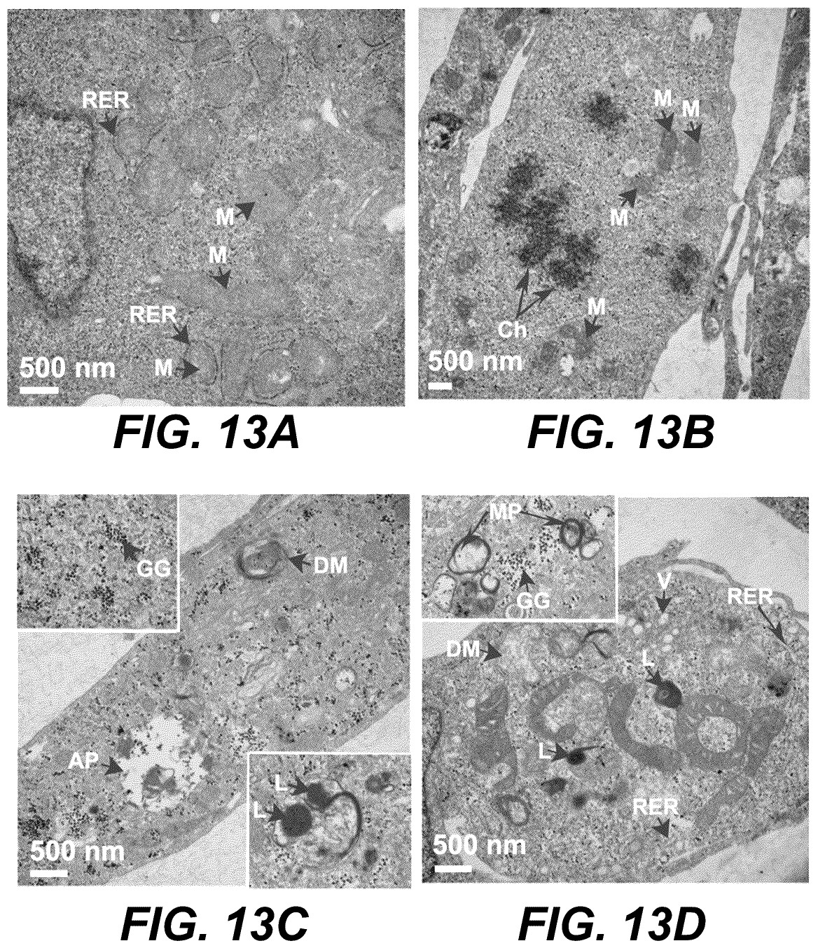

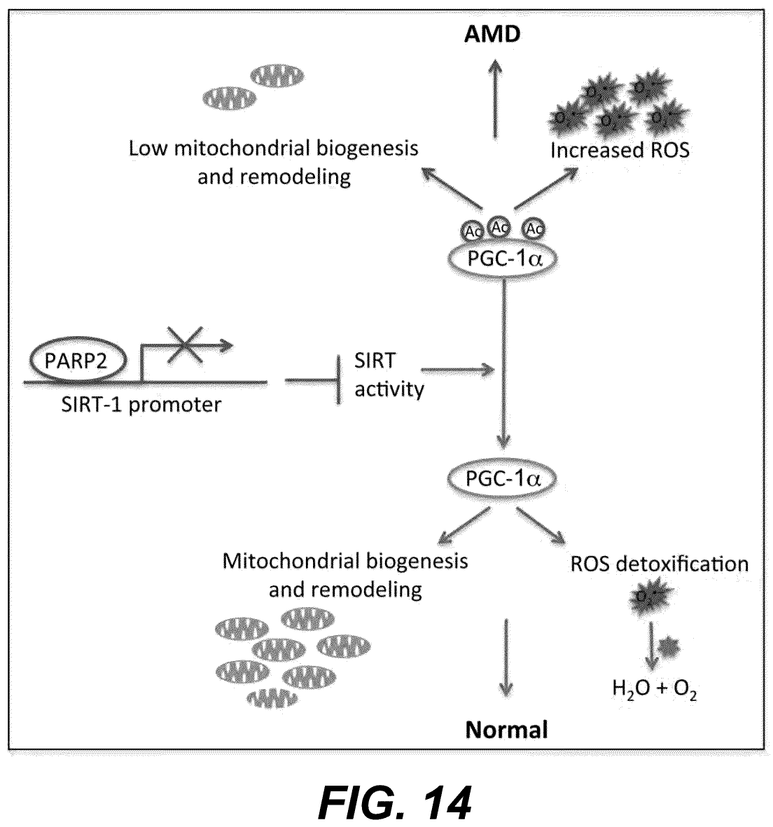

To further analyze the morphology of the ARPE19 CRM-1 overexpressing cells, EM imaging was performed on fixed cells (FIG. 13). Interestingly, the ARPE19 cells overexpressing CRM-1 exhibited pathological phenotypes resembling those identified in dry AMD RPE. Analyses of the CRM-1 overexpressing ARPE19 cells (FIG. 13C, 13D) revealed less dense cytoplasm containing numerous vacuoles and larger glycogen granules forming clumpy aggregates, altered mitochondrial morphology with some mitochondria forming concentric lamellar membranes that coalesce to form outer dense membrane of mitophagosomes, and increased number of mitophagosomes indicative of degenerative mitochondria. In addition, the rough endoplasmic reticulum (RER) appears altered lacking ribosomes, and there was an increased number of lysosomes in the CRM-1 overexpressing ARPE19 (FIGS. 13C, 13D), compared to ARPE19 control cells (FIGS. 13A, 13B). Together, these observations demonstrate an important role for PGC-1α in dry AMD and identify PGC-1α as a possible biomarker and therapeutic target for treatment of dry AMD pathophysiology.

### Example 2

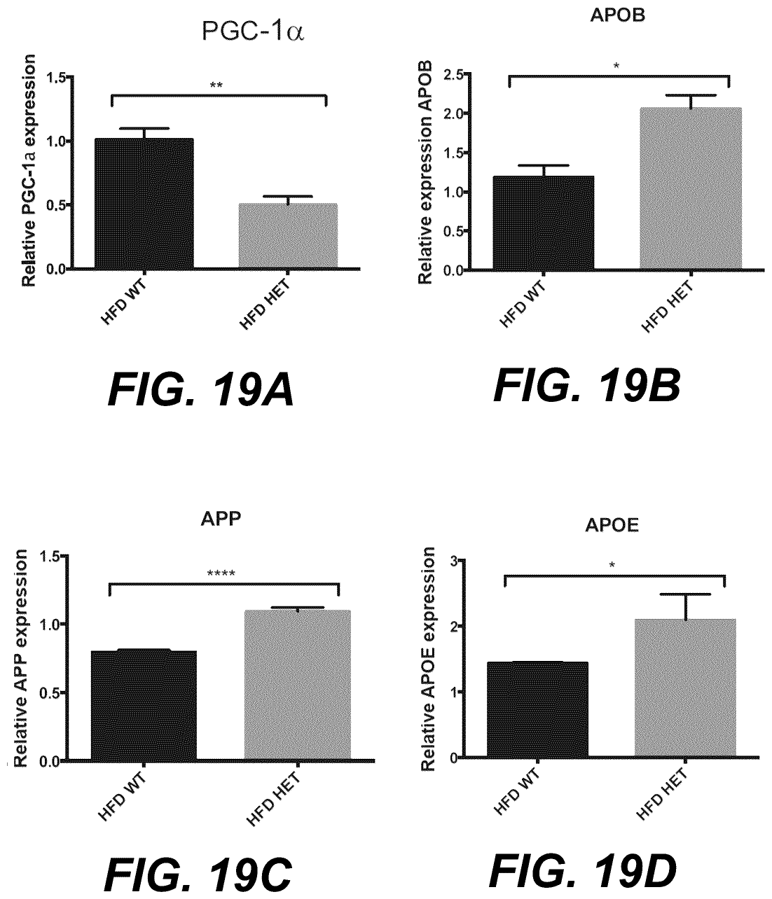

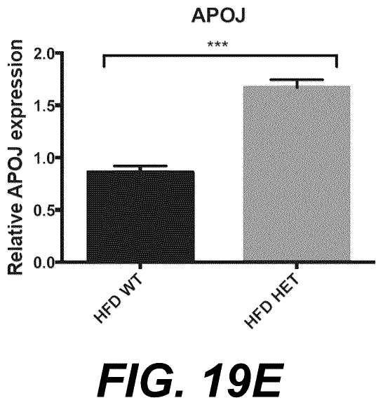

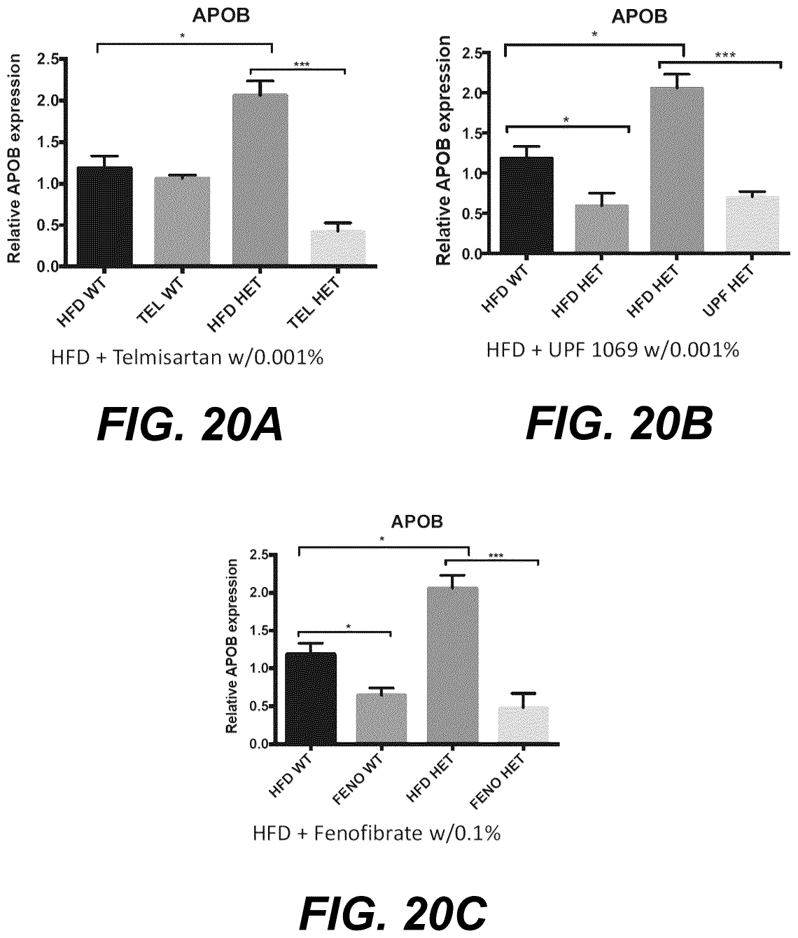

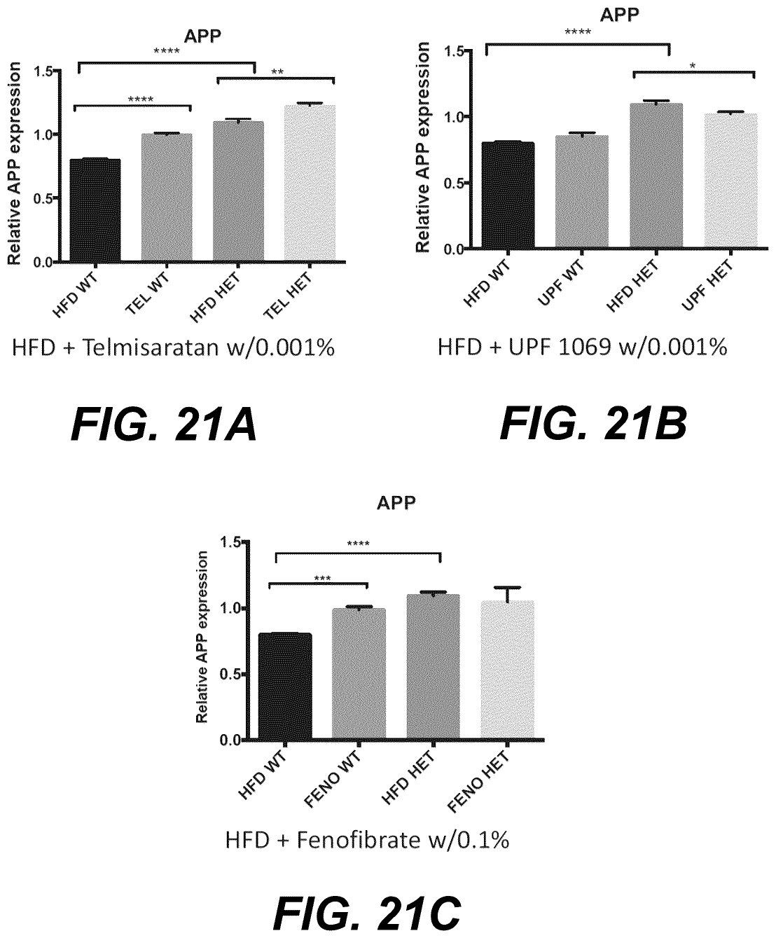

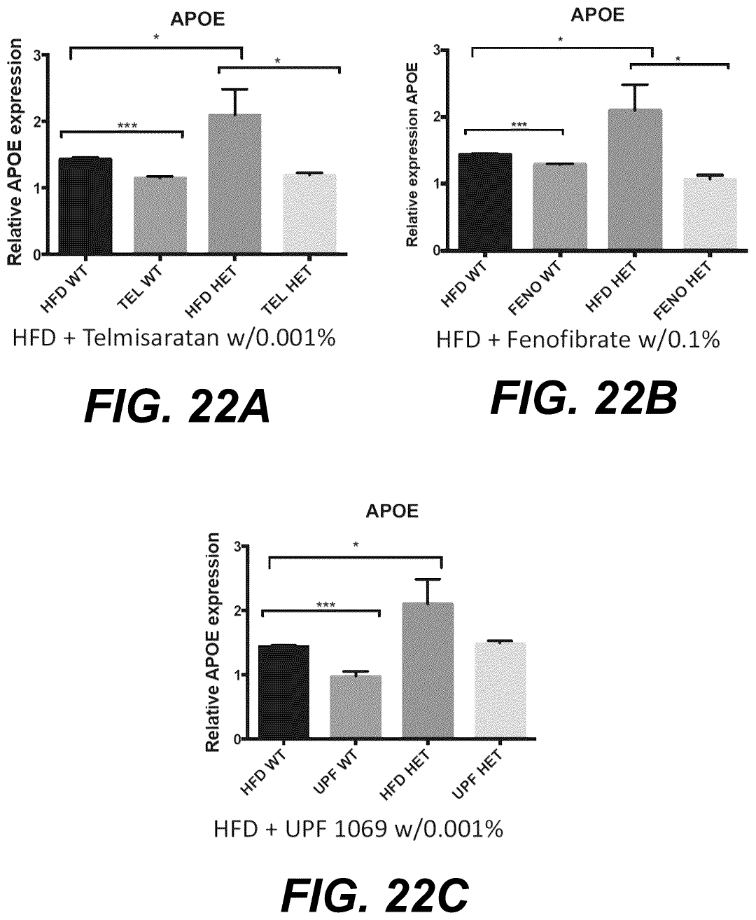

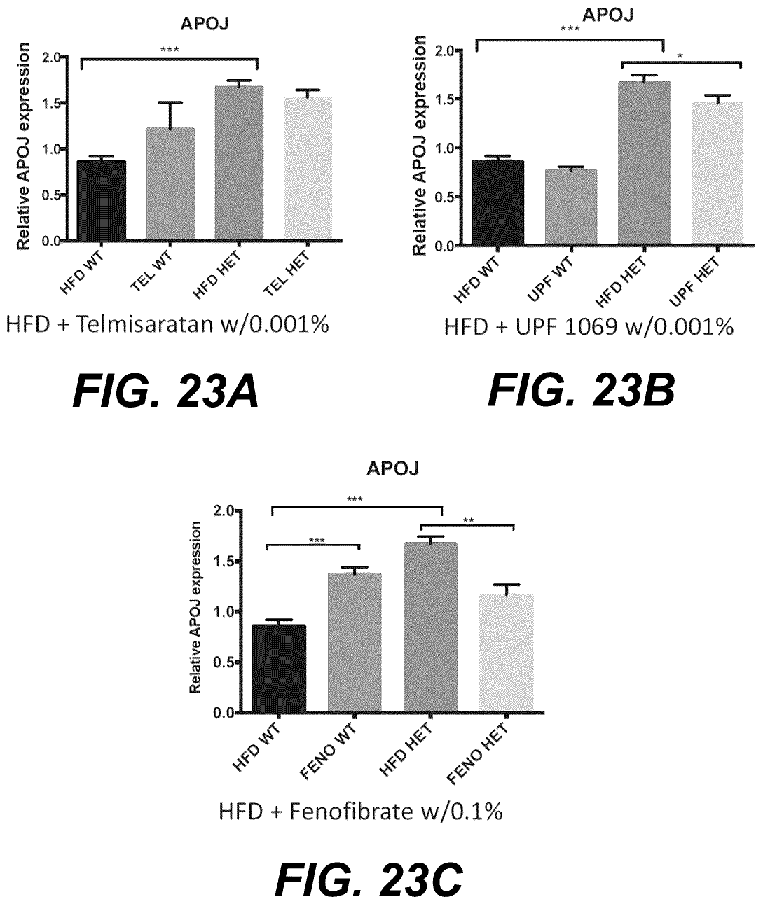

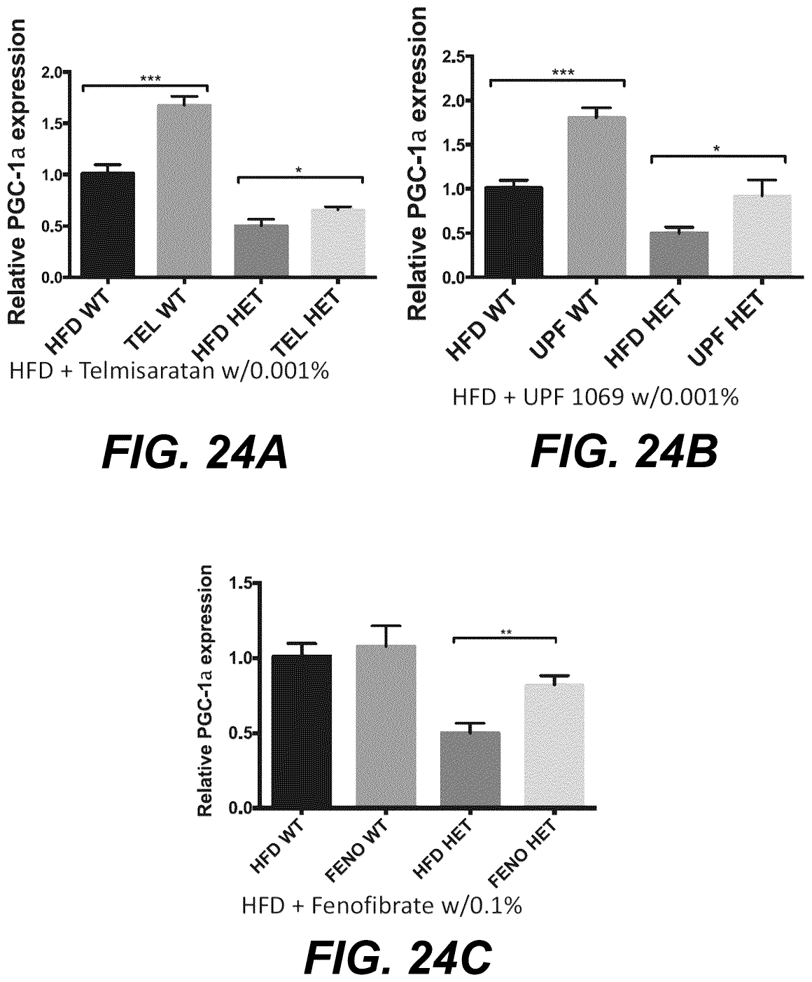

To date, no report has been published on the retinal health and RPE function in mice lacking one allele of PGC-1α, (PGC-1α^{+/-}). High fat diet (HFD) and blue light exposure can induce basal laminar deposits beneath the RPE in mice (Cousins, S., et al., Exp. Eye Res. 75:543-533 (2002), which is incorporated by reference). To first demonstrate that repression in PGC-1α can induce abnormalities in RPE in vivo, the effect of regular and HFD (25% kcal from fat) was investigated on RPE health in PGC-1α^{+/-} mice. Four PGC-1α^{+/-} mice were treated at 8 months of age with HFD (Harlan Laboratories) for 4 months. As control four age-matched PGC-1α^{+/-} mice (“HET”) and four WT mice were treated with isocaloric control diet (RD) (Harlan Laboratories). After four months the animals were sacrificed and EM was performed to verify the RPE morphology and to check for any sign of abnormalities in RPE. FIG. 19 shows differential expression of (A) PGC-1α being repressed, and (B) apolipoprotein B (APOB), (C) amyloid beta precursor protein (APP), (D) apolipoprotein E (APOE), and (E) apolipoprotein J (APOJ) expression being increased in the retina of the HET mice under HFD as compared to that of WT mice.

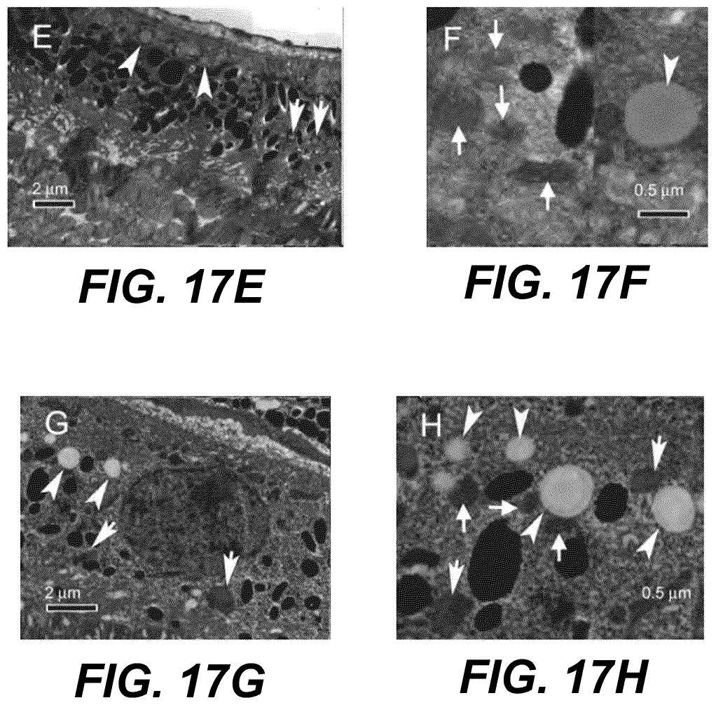

EM expression is repressed in the retina of the PGC-1α+/− (HET) as compared to WT mice. imaging revealed that WT mice fed with regular diet (RD) showed no sings of abnormalities in RPE (FIG. 17A, 17B). In contrary, PGC-1α^{+/-} mice fed with RD showed damaged mitochondria, lipid droplets and lipofuscin in RPE (FIG. 17E, 17F) further demonstrating that repression of PGC-1α can induce disease phenotypes in RPE and retina under normal aging. HFD induced abnormalities both in RPE of WT (FIG. 17C, 17D) and PGC-1α^{+/-} (FIG. 17G, 17H), however, the severity of its impact was significantly greater in PGC-1α^{+/-} RPE (FIG. 6G, 6H, see FIG. 6 legends). In addition, the degree of damage in RPE appeared to be greater in PGC-1α^{+/-} mice fed with HFD (FIG. 17G, 17H) compared to RPE of PGC-1α^{+/-} mice fed with RD (FIG. 17E, 17F). Cytoplasmic degeneration (FIG. 17H), abnormal basal folding and scant melanosomes migrating into the outer segments were also particularly apparent in RPE of PGC-1α^{+/-} mice fed with HFD (data not shown).

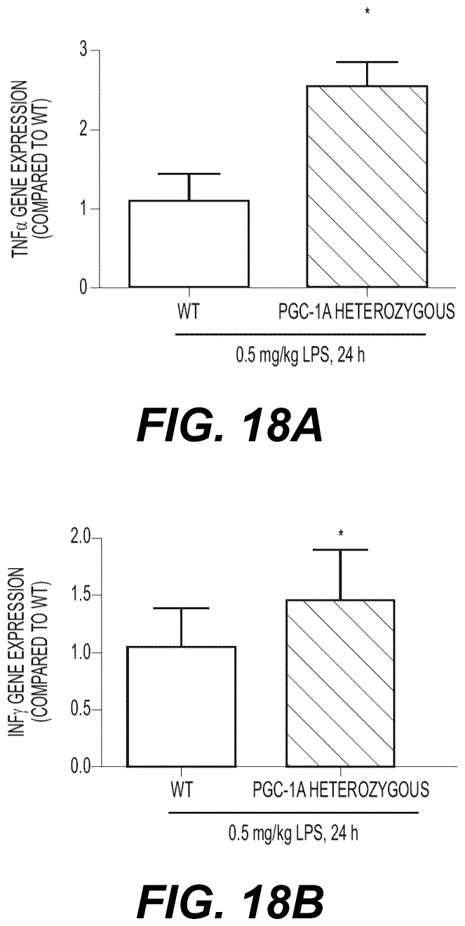

PGC-1α+/−mice exhibit higher inflammation response to LPS injection. It has been reported that PGC-1α reduces inflammation in muscle cells and suppresses a broad inflammatory response. To verify the effect of PGC-1α repression on inflammatory response in retina of the PGC-1α +/−mice, peritoneal lipopolysaccharide (LPS) (0.5 mg/kg, i.p.) injection was performed in 3 PGC-1α +/−mice and 3 age-matched WT mice. After 24 hours the mice were sacrificed and their eyes were enucleated, retina was extracted for RNA purification and the inflammation response was measured by relative expression of TNFα using Real Time PCR. As shown in FIG. 18A, the TNFα expression was significantly higher in retina of PGC-1α+/−mice compared to that of WT mice. These observations are in accordance with the morphological abnormalities observed in PGC-1α RPE and retina.

### Example 3

The HET mice described in Example 2 were administered various compounds to determine the effects of these compounds on the development of AMD. In the High Fat Diet (HFD) experiments, 5 wild type (WT) mice, 5 PGC-1α+/−mice, 5 PGC-1α−/−mice at 2 months old with C57BL/6 background were fed with regular diet (RD) or high fat diet for 16 weeks. The HFD contained 22% calories from fat (Testdiet, 5015). The regular diet was an isocaloric diet that contained 11% calories from fat (Testdiet, 5001).

Every week the body weight was measured. After 4 months the mice were sacrificed, the body weight was measured, the eyes were enucleated, with one eye being fixed with 2.5% glutaraldehyde for electron microscopy imaging. The other eye was dissected; the retina was extracted and processed for protein, RNA and DNA extraction. The liver was also dissected, fixed for sectioning and staining but also for RNA, DNA and protein extraction for biochemical analysis.

To test the effects of telmisartan, fenofibrate and UPF 1069 on mice, 5 wild type (WT) mice, 5 PGC-1α+/−mice, 5 PGC-1α−/−mice at 2 months old were administrated with RD, HFD, or HFD (Testdiet, 5015) containing one of the three compounds. For telmisartan and UPF, 0.001% final concentration of each compound was added to the 5015 diet. Fenofibrate was added at 0.1% final concentration to the 5015 diet. 11 weeks after beginning of administration of the compounds, the mice were sacrificed, the body weight was measured, the eyes were enucleated, with the retinas being extracted and processed for RNA isolation. The liver from each mouse was fixed and processed for sectioning and staining and for RNA and protein extraction.

### REFERENCES

All references cited herein and below are incorporated by reference in their entirety.
Abdelsalam A, Del Priore L, Zarbin M A (1999) Drusen in age-related macular degeneration: pathogenesis, natural course, and laser photocoagulation-induced regression. Survey of ophthalmology 44:1-29.
Ambati J, Fowler B J (2012) Mechanisms of age-related macular degeneration. Neuron 75:26-39.
Beatty S, Koh H, Phil M, Henson D, Boulton M (2000) The role of oxidative stress in the pathogenesis of age-related macular degeneration. Survey of ophthalmology 45:115-134.
Bjorkoy G, Lamark T, Pankiv S, Overvatn A, Brech A, Johansen T (2009) Monitoring autophagic degradation of p62/SQSTM1. Methods in enzymology 452:181-197.
Bjorkoy G, Lamark T, Brech A, Outzen H, Perander M, Overvatn A, Stenmark H, Johansen T (2005) p62/SQSTM1 forms protein aggregates degraded by autophagy and has a protective effect on huntingtin-induced cell death. The Journal of cell biology 171:603-614.
Bok D (1993) The retinal pigment epithelium: a versatile partner in vision. Journal of cell science Supplement 17:189-195.
Boulton M, Dayhaw-Barker P (2001) The role of the retinal pigment epithelium: topographical variation and ageing changes. Eye (Lond) 15:384-389.
Bowes Rickman C, Farsiu S, Toth C A, Klingeborn M (2013) Dry age-related macular degeneration: mechanisms, therapeutic targets, and imaging. Invest Ophthalmol Vis Sci 54:ORSF68-80.
Cai J, Nelson K C, Wu M, Sternberg P, Jr., Jones D P (2000) Oxidative damage and protection of the RPE. Prog Retin Eye Res 19:205-221.
Chen H, Chan D C (2009) Mitochondrial dynamics—fusion, fission, movement, and mitophagy—in neurodegenerative diseases. Human molecular genetics 18:R169-176.
Chen Y, Wang J, Cai J, Sternberg P (2010) Altered mTOR signaling in senescent retinal pigment epithelium. Invest Ophthalmol Vis Sci 51:5314-5319.
Cho Y, Cao X, Shen D, Tuo J, Parver L M, Rickles F R, Chan C C (2011) Evidence for enhanced tissue factor expression in age-related macular degeneration. Laboratory investigation; a journal of technical methods and pathology 91:519-526.
Corum D G, Tsichlis P N, Muise-Helmericks R C (2013) AKT3 controls mitochondrial biogenesis and autophagy via regulation of the major nuclear export protein CRM-1. FASEB journal: official publication of the Federation of American Societies for Experimental Biology.
Czaja M J (2010) Autophagy in health and disease. 2. Regulation of lipid metabolism and storage by autophagy: pathophysiological implications. American journal of physiology Cell physiology 298:C973-978.
De Duve C (1963) The lysosome. Scientific American 208:64-72.
De Duve C, Wattiaux R (1966) Functions of lysosomes. Annual review of physiology 28:435-492.
Eisele, P., et al., J. Biol. Chem., 288:2246-2260 (2013)
Ferris F L, 3rd, Fine S L, Hyman L (1984) Age-related macular degeneration and blindness due to neovascular maculopathy. Arch Ophthalmol 102:1640-1642.
Field M G, corner G M, Kawaji T, Petty H R, Elner V M (2012) Noninvasive imaging of mitochondrial dysfunction in dry age-related macular degeneration. Ophthalmic surgery, lasers & imaging: the official journal of the International Society for Imaging in the Eye 43:358-365.
Finn P F, Dice J F (2006) Proteolytic and lipolytic responses to starvation. Nutrition 22:830-844.
Gehrs K M, Anderson D H, Johnson L V, Hageman G S (2006) Age-related macular degeneration—emerging pathogenetic and therapeutic concepts. Ann Med 38:450-471.
Gottlieb R A, Carreira R S (2010) Autophagy in health and disease. 5. Mitophagy as a way of life. American journal of physiology Cell physiology 299:C203-210.
Gu X, Neric N J, Crabb J S, Crabb J W, Bhattacharya S K, Rayborn M E, Hollyfield J G, Bonilha V L (2012) Age-related changes in the retinal pigment epithelium (RPE). PLoS One 7:e38673.
Gwinn D M, Shackelford D B, Egan D F, Mihaylova M M, Mery A, Vasquez D S, Turk B E, Shaw R J (2008) AMPK phosphorylation of raptor mediates a metabolic checkpoint. Molecular cell 30:214-226.
Handschin, C. and Spiegelman, B., Nature, 454:463-469 (2008)
Johnson S C, Rabinovitch P S, Kaeberlein M (2013) mTOR is a key modulator of ageing and age-related disease. Nature 493:338-345.
Kaarniranta K, Sinha D, Blasiak J, Kauppinen A, Vereb Z, Salminen A, Boulton M E, Petrovski G (2013) Autophagy and heterophagy dysregulation leads to retinal pigment epithelium dysfunction and development of age-related macular degeneration. Autophagy 9:973-984.
Kim D H, Sarbassov D D, Ali S M, Latek R R, Guntur K V, Erdjument-Bromage H, Tempst P, Sabatini D M (2003) GbetaL, a positive regulator of the rapamycin-sensitive pathway required for the nutrient-sensitive interaction between raptor and mTOR. Molecular cell 11:895-904.
Kim J, Kundu M, Viollet B, Guan K L (2011) AMPK and mTOR regulate autophagy through direct phosphorylation of Ulk1. Nat Cell Biol 13:132-141.
Kimmelman A C (2011) The dynamic nature of autophagy in cancer. Genes & development
Kinnunen K, Petrovski G, Moe M C, Berta A, Kaarniranta K (2012) Molecular mechanisms of retinal pigment epithelium damage and development of age-related macular degeneration. Acta ophthalmologica 90:299-309.
Klein R, Klein B E, Lee K E, Cruickshanks K J, Gangnon R E (2006) Changes in visual acuity in a population over a 15-year period: the Beaver Dam Eye Study. American journal of ophthalmology 142:539-549.
Klein R, Chou C F, Klein B E, Zhang X, Meuer S M, Saaddine J B (2011) Prevalence of age-related macular degeneration in the US population. Arch Ophthalmol 129:75-80.
Knorr H L, Linde-Behringer M, Gossler B, Mayer U M (1993) Human retinal pigment epithelium in vitro: influence of low oxygen tension, glucose and insulin. Ophthalmic research 25:226-234.
Kokkinaki M, Abu-Asab M, Gunawardena N, Ahern G, Javidnia M, Young J, Golestaneh N (2013) Klotho regulates retinal pigment epithelial functions and protects against oxidative stress. The Journal of neuroscience: the official journal of the Society for Neuroscience 33:16346-16359.
Komatsu M et al. (2007) Homeostatic levels of p62 control cytoplasmic inclusion body formation in autophagy-deficient mice. Cell 131:1149-1163.
Kozlowski M R (2012) RPE cell senescence: a key contributor to age-related macular degeneration. Medical hypotheses 78:505-510.
Kurz D J, Decary S, Hong Y, Erusalimsky J D (2000) Senescence-associated (beta)-galactosidase reflects an increase in lysosomal mass during replicative ageing of human endothelial cells. Journal of cell science 113 (Pt 20):3613-3622.
Kuusisto E, Kauppinen T, Alafuzoff I (2008) Use of p62/SQSTM1 antibodies for neuropathological diagnosis. Neuropathology and applied neurobiology 34:169-180.
Laplante M, Sabatini D M (2012) mTOR signaling in growth control and disease. Cell 149:274-293.
Lee J, Giordano S, Zhang J (2012) Autophagy, mitochondria and oxidative stress: cross-talk and redox signalling. The Biochemical journal 441:523-540.
Lim L S, Mitchell P, Seddon J M, Holz F G, Wong T Y (2012) Age-related macular degeneration. Lancet 379:1728-1738.
Lin H, Xu H, Liang F Q, Liang H, Gupta P, Havey A N, Boulton M E, Godley B F (2011) Mitochondrial DNA damage and repair in RPE associated with aging and age-related macular degeneration. Invest Ophthalmol Vis Sci 52:3521-3529.
Maminishkis A, Chen S, Jalickee S, Banzon T, Shi G, Wang F E, Ehalt T, Hammer J A, Miller S S (2006) Confluent monolayers of cultured human fetal retinal pigment epithelium exhibit morphology and physiology of native tissue. Invest Ophthalmol Vis Sci 47:3612-3624.
Matsunaga H, Handa J T, Aotaki-Keen A, Sherwood S W, West M D, Hjelmeland L M (1999) Beta-galactosidase histochemistry and telomere loss in senescent retinal pigment epithelial cells. Invest Ophthalmol Vis Sci 40:197-202.
Mitter S K, Rao H V, Qi X, Cai J, Sugrue A, Dunn W A, Jr., Grant M B, Boulton M E (2012) Autophagy in the retina: a potential role in age-related macular degeneration. Advances in experimental medicine and biology 723:83-90.
Mizushima N (2004) Methods for monitoring autophagy. The international journal of biochemistry & cell biology 36:2491-2502.
Mizushima N, Yoshimori T (2007) How to interpret LC3 immunoblotting. Autophagy 3:542-545.
Murphy M P (2013) Mitochondrial Dysfunction Indirectly Elevates ROS Production by the Endoplasmic Reticulum. Cell metabolism 18:145-146.
Nazio F, Strappazzon F, Antonioli M, Bielli P, Cianfanelli V, Bordi M, Gretzmeier C, Dengjel J, Piacentini M, Fimia G M, Cecconi F (2013) mTOR inhibits autophagy by controlling ULK1 ubiquitylation, self-association and function through AMBRA1 and TRAF6. Nat Cell Biol 15:406-416.
Negi A, Marmor M F (1984) Experimental serous retinal detachment and focal pigment epithelial damage. Arch Ophthalmol 102:445-449.
Nishikawa T, Bellance N, Damm A, Bing H, Zhu Z, Handa K, Yovchev M I, Sehgal V, Moss T J, Oertel M, Ram P T, Pipinos, II, Soto-Gutierrez A, Fox I J, Nagrath D (2014) A switch in the source of ATP production and a loss in capacity to perform glycolysis are hallmarks of hepatocyte failure in advance liver disease. Journal of hepatology 60:1203-1211.
Notomi S, Hisatomi T, Murakami Y, Terasaki H, Sonoda S, Asato R, Takeda A, Ikeda Y, Enaida H, Sakamoto T, Ishibashi T (2013) Dynamic increase in extracellular ATP accelerates photoreceptor cell apoptosis via ligation of P2RX7 in subretinal hemorrhage. PLoS One 8:e53338.
Nowak J Z (2006) Age-related macular degeneration (AMD): pathogenesis and therapy. Pharmacological reports: PR 58:353-363.
Orenstein S J, Kuo S H, Tasset I, Arias E, Koga H, Fernandez-Carasa I, Cortes E, Honig L S, Dauer W, Consiglio A, Raya A, Sulzer D, Cuervo A M (2013) Interplay of LRRK2 with chaperone-mediated autophagy. Nature neuroscience 16:394-406.
Pankiv S, Clausen T H, Lamark T, Brech A, Bruun J A, Outzen H, Overvatn A, Bjorkoy G, Johansen T (2007) p62/SQSTM1 binds directly to Atg8/LC3 to facilitate degradation of ubiquitinated protein aggregates by autophagy. The Journal of biological chemistry 282:24131-24145.
Pennesi M E, Neuringer M, Courtney R J (2012) Animal models of age related macular degeneration. Molecular aspects of medicine 33:487-509.
Rein D B, Wittenborn J S, Zhang X, Honeycutt A A, Lesesne S B, Saaddine J (2009) Forecasting age-related macular degeneration through the year 2050: the potential impact of new treatments. Arch Ophthalmol 127:533-540.
Rose S, Frye R E, Slattery J, Wynne R, Tippett M, Pavliv O, Melnyk S, James S J (2014) Oxidative stress induces mitochondrial dysfunction in a subset of autism lymphoblastoid cell lines in a well-matched case control cohort. PLoS One 9:e85436.
Rubinsztein D C, Marino G, Kroemer G (2011) Autophagy and aging. Cell 146:682-695.
Schmitz-Valckenberg S, Fleckenstein M, Scholl H P, Holz F G (2009) Fundus autofluorescence and progression of age-related macular degeneration. Survey of ophthalmology 54:96-117.
Sena L A, Chandel N S (2012) Physiological roles of mitochondrial reactive oxygen species. Molecular cell 48:158-167.
Shelton D N, Chang E, Whittier P S, Choi D, Funk W D (1999) Microarray analysis of replicative senescence. Current biology: CB 9:939-945.
Singh R, Kaushik S, Wang Y, Xiang Y, Novak I, Komatsu M, Tanaka K, Cuervo
A M, Czaja M J (2009) Autophagy regulates lipid metabolism. Nature 458:1131-1135.
Strauss O (2005) The retinal pigment epithelium in visual function. Physiological reviews 85:845-881.
Suzuki A, Kusakai G, Kishimoto A, Shimojo Y, Ogura T, Lavin M F, Esumi H (2004) IGF1 phosphorylates AMPK-alpha subunit in ATM-dependent and LKB1-independent manner. Biochem Biophys Res Commun 324:986-992.
Tanida I, Minematsu-Ikeguchi N, Ueno T, Kominami E (2005) Lysosomal turnover, but not a cellular level, of endogenous LC3 is a marker for autophagy. Autophagy 1:84-91.
Troncoso R, Vicencio J M, Parra V, Nemchenko A, Kawashima Y, Del Campo A, Toro B, Battiprolu P K, Aranguiz P, Chiong M, Yakar S, Gillette T G, Hill J A, Abel E D, Leroith D, Lavandero S (2012) Energy-preserving effects of IGF1 antagonize starvation-induced cardiac autophagy. Cardiovascular research 93:320-329.
Vessey K A, Greferath U, Aplin F P, Jobling A I, Phipps J A, Ho T, De longh R U, Fletcher E L (2014) Adenosine triphosphate-induced photoreceptor death and retinal remodeling in rats. The Journal of comparative neurology.
Viiri J, Amadio M, Marchesi N, Hyttinen J M, Kivinen N, Sironen R, Rilla K, Akhtar S, Provenzani A, D'Agostino VG, Govoni S, Pascale A, Agostini H, Petrovski G, Salminen A, Kaarniranta K (2013) Autophagy activation clears ELAVL1/HuR-mediated accumulation of SQSTM1/p62 during proteasomal inhibition in human retinal pigment epithelial cells. PLoS One 8:e69563.
Wang A L, Lukas T J, Yuan M, Du N, Tso M O, Neufeld A H (2009) Autophagy and exosomes in the aged retinal pigment epithelium: possible relevance to drusen formation and age-related macular degeneration. PLoS One 4:e4160.
Wang J, Ohno-Matsui K, Morita I (2012) Elevated amyloid beta production in senescent retinal pigment epithelium, a possible mechanism of subretinal deposition of amyloid beta in age-related macular degeneration. Biochem Biophys Res Commun 423:73-78.
Wang Y, Singh R, Xiang Y, Czaja M J (2010) Macroautophagy and chaperone-mediated autophagy are required for hepatocyte resistance to oxidant stress. Hepatology 52:266-277.
Wright G L, Maroulakou I G, Eldridge J, Liby T L, Sridharan V, Tsichlis P N, Muise-Helmericks R C (2008) VEGF stimulation of mitochondrial biogenesis: requirement of AKT3 kinase. FASEB journal: official publication of the Federation of American Societies for Experimental Biology 22:3264-3275.
Yang P, Baciu P, Kerrigan B C, Etheridge M, Sung E, Toimil B A, Berchuck J E, Jaffe G J (2014) Retinal Pigment Epithelial Cell Death by the Alternative Complement Cascade: Role of Membrane Regulatory Proteins, Calcium, PKC, and Oxidative Stress. Invest Ophthalmol Vis Sci 55:3012-3021.
Yu L, McPhee C K, Zheng L, Mardones G A, Rong Y, Peng J, Mi N, Zhao Y, Liu Z, Wan F, Hailey D W, Oorschot V, Klumperman J, Baehrecke E H, Lenardo M J (2010) Termination of autophagy and reformation of lysosomes regulated by mTOR. Nature 465:942-946.
Zatloukal K, Stumptner C, Fuchsbichler A, Heid H, Schnoelzer M, Kenner L, Kleinert R, Prinz M, Aguzzi A, Denk H (2002) p62 Is a common component of cytoplasmic inclusions in protein aggregation diseases. The American journal of pathology 160:255-263.
Zhang Y, Qi H, Taylor R, Xu W, Liu LF, Jin S (2007) The role of autophagy in mitochondria maintenance: characterization of mitochondrial functions in autophagy-deficient S. cerevisiae strains. Autophagy 3:337-346.

1. A method of determining if a subject is at risk of developing dry age-related macular degeneration (dry AMD), the method comprising
a) determining expression or activity levels of at least one of peroxisome proliferator-activated receptor-gamma coactivator 1-alpha (PGC-1α), NAD-dependent deacetylase sirtuin-1 (SIRT-1), and Poly (Adenosine diphosphate-Ribose) Polymerase 2 (PARP2) in a sample obtained from the subject; or determining mRNA levels of RAC-gamma serine/threonine-protein kinase (AKT3) in a sample obtained from the subject,
b) comparing the determined expression or activity levels of PGC-1α or SIRT-1 or PARP2 in the subject with the expression or activity levels obtained from subjects determined to define normal expression or activity levels of PGC-1α or SIRT-1 or PARP2 to determine if the subject's expression or activity levels of PGC-1α or SIRT-1 or PARP2 are altered compared to normal expression or activity levels of PGC-1α or SIRT-1 or PARP2; or comparing the determined mRNA levels of AKT3 in the subject with the mRNA levels obtained from subjects determined to define normal mRNA levels of AKT3 to determine if the subject's mRNA levels of AKT3 are altered compared to normal mRNA levels of AKT3,
wherein a reduction in the expression or activity levels of PGC-1α or SIRT-1 in the sample compared to normal levels is indicative that the subject is at risk of developing dry AMD; an increase in the expression or activity levels of PARP2 in the sample compared to normal levels is indicative that the subject is at risk of developing dry AMD; and a reduction in the mRNA levels of AKT3 in the sample compared to normal mRNA levels is indicative that the subject is at risk of developing dry AMD.

2. The method of claim 1, wherein the sample is obtained from a skin biopsy or is a blood sample.

3. The method claim 2, wherein the method comprises obtaining the skin biopsy from the subject and transforming the skin fibroblasts obtained from the skin biopsy into retinal pigment epithelial cells (RPE) prior to determining the expression or activity levels of the PGC-1α or SIRT-1 or PARP2 in the sample, or prior to determining the mRNA levels of the AKT3 in the sample.

4-13. (canceled)

14. A method of monitoring the progression of dry age-related macular degeneration (dry AMD) in a subject, the method comprising
a) analyzing at least two samples from the subject with each sample taken at different time points to determine expression or activity levels of at least one of peroxisome proliferator-activated receptor-gamma coactivator 1-alpha (PGC-1α), NAD-dependent deacetylase sirtuin-1 (SIRT-1), and Poly (Adenosine diphosphate-Ribose) Polymerase 2 (PARP2); or analyzing at least two samples from the subject with each sample taken at different time points to determine mRNA levels of RAC-gamma serine/threonine-protein kinase (AKT3), and
b) comparing the expression or activity levels of the subject's PGC-1α or SIRT-1 or PARP2 over time to determine if expression or activity levels of PGC-1α or SIRT-1 or PARP2 are changing over time; comparing the mRNA levels of the subject's AKT3 over time to determine if mRNA levels of AKT3 are changing over time,
wherein a decrease in the subject's expression or activity levels of PGC-1α or SIRT-1 over time is indicative that the subject's risk of suffering from dry AMD is increasing over time; wherein an increase in the subject's expression or activity levels of PARP2 over time is indicative that the subject's risk of suffering from dry AMD is increasing over time; and wherein a decrease in the subject's mRNA levels of AKT3 over time is indicative that the subject's risk of suffering from dry AMD is increasing over time.

15-25. (canceled)

26. A method of reversing symptoms of dry age-related macular degeneration (dry AMD) in a subject in need of treatment thereof, the method comprising administering at least one of (i) a compound that induces expression or activity of peroxisome proliferator-activated receptor-gamma coactivator 1-alpha (PGC-1α) in retinal pigment epithelial cells; (ii) a compound that induces expression of RAC-gamma serine/threonine-protein kinase (AKT3) in retinal pigment epithelial cells; (iii) a compound that induces expression of NAD-dependent deacetylase sirtuin-1 (SIRT-1) in retinal pigment epithelial cells; and (iv) an inhibitor of Poly (Adenosine diphosphate-Ribose) Polymerase 2 (PARP2) in a therapeutically effective amount.

27. The method of claim 26, wherein the compound that induces expression of PGC-1α is a fibrate.

28. The method of claim 27, wherein the fibrate is selected from the group consisting of bezafibrate, ciprofibrate, clofibrate gemfibrozil, fenofibrate and clinofibrate.

29. The method of claim 26 wherein the compound that induces expression of PGC-1α is a sartan.

30. The method of claim 29, wherein the sartan is selected from the group consisting of losartan, telmisartan, irbesartan, azilsartan, olmesartan, valsartan, eprosartan, temposartan and candesartan.

31-34. (canceled) (Currently amended) The method of claim 26, wherein the PARP inhibitor is administered topically.

36. The method of claim 35, wherein the PARP inhibitor is comprised within a liquid composition.

37. The method of claim 36, wherein the amount of PARP inhibitor that is administered to the subject is about 1 ng.

38. The method of claim 26, further comprising determining expression or activity levels of PGC-1α in a sample obtained from the subject, and administering the compound that induces expression or activity of PGC-1α when there is a reduction in the expression or activity levels of PGC-1α in the sample compared to normal levels of PGC-1α.

39. The method of claim 38, wherein the sample is obtained from a skin biopsy.

40. The method claim 39, wherein the method comprises obtaining the skin biopsy from the subject and transforming the skin fibroblasts obtained from the skin biopsy into retinal pigment epithelial cells (RPE) prior to determining the expression or activity levels of the PGC-1α in the sample.
